(19) United States
(12) Patent Application Publication
(10) Pub. No.: US 2009/0258412 A1
(43) Pub. Date: Oct. 15, 2009

(54) REACTOR

(75) Inventors: Toshiki Moriwaki, Kanagawa (JP); Takanori Anaguchi, Kanagawa (JP)

Correspondence Address: WOLF GREENFIELD & SACKS, P.C., 600 ATLANTIC AVENUE, BOSTON, MA, 02210-2206, US

(73) Assignee: Sony Corporation, Tokyo (JP)

(21) Appl. No.: 12/422,547

(22) Filed: Apr. 13, 2009

(30) Foreign Application Priority Data
Apr. 15, 2008 (JP) 2008-105841

(51) Int. Cl. C12M 1/36

(52) U.S. Cl. 435/286.1

(57) ABSTRACT

Disclosed herein is a reactor, including, a plurality of reaction regions, a plurality of heating elements, each arranged in each of the reaction regions, and cooling elements that cool other regions than reaction regions which are heated by the heating elements, wherein the heating element including a heater and a temperature detecting element and having a detection section configured to detect temperature from the temperature detecting element and a temperature control section configured to control the heater's temperature according to the detected temperature information.

### BACKGROUND OF THE INVENTION

1. Field of the Invention

The present invention relates to a reactor to be applied to PCR for gene amplification and, more particularly, to a reactor capable of accurate temperature control.

2. Description of the Related Art

In the case where it is necessary to control reactions according to temperature conditions, it is desirable to be able to control the temperature conditions more accurately. Capability of accurate temperature control is desirable for any reactors for liquids, solids, and gases. This holds true in the technical field of gene analysis.

One example of such cases is PCR (polymerase chain reaction) for gene amplification. PCR may be regarded as the standard process for quantitative analysis of nucleic acid in trace amounts.

PCR is designed to repeat the cycle of amplification, which consists of “thermal denaturation→annealing with primer→polymerase extension reaction”, thereby amplifying the amount of DNA several hundred thousand times.

The PCR amplified product obtained in this manner can be monitored in real time for quantitative analysis of nucleic acid in trace amounts.

However, PCR requires that the amplification cycle be accurately controlled. To this end, a highly accurate temperature control is essential.

Inadequate temperature control will lead to amplification of unnecessary DNA sequence or prevent amplification.

Thus, the above-mentioned reactor needs capability of highly accurate temperature control as a reactor. Technologies relating to this are disclosed in Japanese Patent Laid-open No. 2003-298068 and Japanese Patent Laid-open No. 2004-025426.

Control of heat generation in a minute region is accomplished by means of semiconductor devices. Semiconductor devices can be applied to heater elements arranged in matrix form. The technologies relating to matrix arrangement and reaction control are disclosed in Japanese Patent Laid-open Nos. 2003-180328 and 2006-238759, respectively.

### SUMMARY OF THE INVENTION

The reactor poses a problem of causing heat diffusion from adjacent heaters if it is provided with semiconductor elements or resistance heating elements for heat control in minute regions arranged in a matrix pattern.

FIG. 1 shows how heat diffusion takes place.

When three heaters (A)1, (B)2, and (C)3 are turned on simultaneously, there exists a temperature profile between the heaters (A)1 and (B)2, as shown in FIG. 1. It is noted that heat diffusion raises the temperature at an intermediate point X between the heaters (A)1 and (B)2. It is also noted that peak B is higher than peak A, which are peak temperatures due to heaters (B)2 and (A)1, respectively. This is because the heater (B)2 is affected by heat diffusion from the heaters (A)1 and (C)3.

Heat diffusion poses the following problems.
The actual temperature is higher than the temperature which has been set for the heater. This prevents accurate temperature control.
Increasing the distance between adjacent heaters to avoid heat diffusion increases the total area of the matrix.
Individual temperature control of heaters arranged in a matrix pattern is difficult to achieve because heaters vary in heat diffusion depending on their positions.

An embodiment of the present invention to provide a reactor which is capable of accurate temperature control even though heat diffusion from adjacent heaters takes place.

According to an embodiment of the present invention there is provided a reactor, including: a plurality of reaction regions;

a plurality of heating elements, each arranged in each of the reaction regions; and

cooling elements that cool other regions than reaction regions which are heated by the heating elements, wherein

the heating element including a heater and a temperature detecting element and having
detection means for detecting temperature from the temperature detecting element and
temperature control means for controlling the heater's temperature according to the detected temperature information,
the temperature control means performing
   processing for the temperature cycle which includes the first temperature holding control in denature treatment,
   processing for the second temperature holding control in cooling from denature treatment to annealing treatment and also in annealing treatment,
   processing for the first temperature rise control for the first heating from annealing treatment to extension treatment,
   processing for the third temperature holding control in extension treatment, and
   processing for the second temperature rise control for the second heating from extension treatment to denature treatment.

According to another embodiment of the present invention there is provided a reactor, including:

a plurality of reaction regions;

a plurality of heating elements, each arranged in each of the reaction regions; and

cooling elements that cool other regions than reaction regions which are heated by the heating elements, wherein

the heating element including a heater and a temperature detecting element and having
detection means for detecting temperature from the temperature detecting element and
temperature control means for controlling the heater's temperature according to the detected temperature information,
the temperature control means performing processing includes
   a first temperature holding control in denature treatment,
   a temperature down control in cooling from denature treatment to annealing treatment,
   a second temperature holding control in annealing treatment and extension treatment, and
   a temperature rise control in heating from extension treatment to denature treatment.

The present invention offers the advantage of performing accurate temperature control even when heat diffusion from adjacent heaters occurs.

### BRIEF DESCRIPTION OF THE DRAWINGS

FIG. 1 is a diagram showing an example of heat diffusion;

FIG. 2 is a conceptual diagram showing the reactor according to an embodiment of the present invention;

FIG. 3 is a schematic diagram showing the structure of the heating part in the reactor according to the embodiment of the present invention;

FIG. 4 is a schematic diagram showing the structure of the system to perform temperature control feedback for the control unit in the reactor according to the embodiment of the present invention;

FIG. 5 shows one example of the control parameters used in this embodiment;

FIG. 6 is a flow chart to illustrate the basic feed back control in this embodiment;

FIG. 7 is a flow chart to illustrate the PCR process control;

FIG. 8 is a diagram listing the control processes (phases) in this embodiment;

FIG. 9 is a flow chart to illustrate the action of the phase to set the potentiometer for temperature measurement;

FIG. 10 is a flow chart to illustrate the action of the phase to acquire AD data;

FIG. 11 is a flow chart to illustrate the action of the phase to calculate the amount of heater control;

FIG. 12 shows one example of the control subphases in this embodiment;

FIG. 13 is a flow chart to illustrate the action of the Peltier control phase;

FIG. 14 is a flow chart to illustrate the action of the heater control phase;

FIG. 15 is a schematic diagram showing the structure of the heater matrix device according to the embodiment of the present invention;

FIG. 16 is a circuit diagram showing a first example of the structure of the heater unit in the heater matrix device according to the embodiment of the present invention;

FIG. 17 is a circuit diagram showing one activated state of the circuit shown in FIG. 16;

FIG. 18 is a circuit diagram showing another activated state of the circuit shown in FIG. 16;

FIG. 19 is a circuit diagram showing a modified example of the circuit shown in FIG. 16;

FIG. 20 is a circuit diagram showing another modified example of the circuit shown in FIG. 16;

FIG. 21 is a circuit diagram showing further another modified example of the circuit shown in FIG. 9;

FIG. 22 is a circuit diagram showing further another modified example of the circuit shown in FIG. 16;

FIG. 23 is a circuit diagram showing a typical example of the circuit shown in FIG. 16;

FIG. 24 is a circuit diagram showing a modified example of the circuit shown in FIG. 16;

FIG. 25 is a circuit diagram showing another modified example of the circuit shown in FIG. 9;

FIG. 26 is a schematic diagram showing the structure of the heater matrix device having the heater unit shown in FIG. 18;

FIG. 27 is a circuit diagram showing another modified example of the circuit shown in FIG. 9;

FIG. 28 is a circuit diagram showing further another modified example of the circuit shown in FIG. 9;

FIG. 29 is a schematic diagram showing the structure of the temperature detecting matrix device according to the embodiment of the present invention;

FIG. 30 is a circuit diagram showing the structure of the temperature detecting unit according to the embodiment of the present invention;

FIG. 31 is a graph showing the dependence of dark current on temperature;

FIG. 32 is a graph showing how temperature depends on the forward voltage of the PIN diode which is produced when the PIN diode is given a certain forward current;

FIG. 33 is a schematic diagram showing the structure of the fluorescence detecting matrix device according to the embodiment of the present invention;

FIG. 34 is a circuit diagram showing the structure of the fluorescence detecting unit according to the embodiment of the present invention;

FIG. 35 is a schematic diagram showing the structure of the heater temperature detecting matrix device according to the embodiment of the present invention;

FIG. 36 is a circuit diagram showing the structure of the heater temperature detecting unit according to the embodiment of the present invention;

FIG. 37 is a graph showing the relation between the current of the heater unit and the voltage detected in response to current flowing through the PIN diode of the temperature detecting unit;

FIG. 38 is a schematic diagram showing the structure of the temperature fluorescence detecting matrix device according to the embodiment of the present invention;

FIG. 39 is a circuit diagram showing the structure of the temperature fluorescence detecting unit according to the embodiment of the present invention;

FIG. 40 shows how the temperature fluorescence detecting unit according to the embodiment of the present invention performs temperature detection and fluorescence detection depending on whether the transistors as switches turn on and off;

FIG. 41 is a diagram illustrating how temperature detection is performed by the temperature fluorescence detecting unit according to the embodiment of the present invention;

FIG. 42 is a diagram illustrating how fluorescence detection is performed by the temperature fluorescence detecting unit according to the embodiment of the present invention;

FIG. 43 is a diagram illustrating the fluorescence detection;

FIG. 44 is a schematic diagram showing the structure of the heater temperature fluorescence detecting matrix device according to the embodiment of the present invention; and

FIG. 45 is a circuit diagram showing the structure of the heater temperature fluorescence detecting unit according to the embodiment of the present invention.

### DETAILED DESCRIPTION OF THE PREFERRED EMBODIMENTS

The embodiments of the present invention will be described below with reference to the accompanying drawings.

The embodiments illustrated in the accompanying drawings represent merely some typical ones of the present invention, and they should not be construed to restrict the scope of the present invention.

The drawings used hereunder show the structure of the apparatus in a simplified manner for the convenience of illustration.

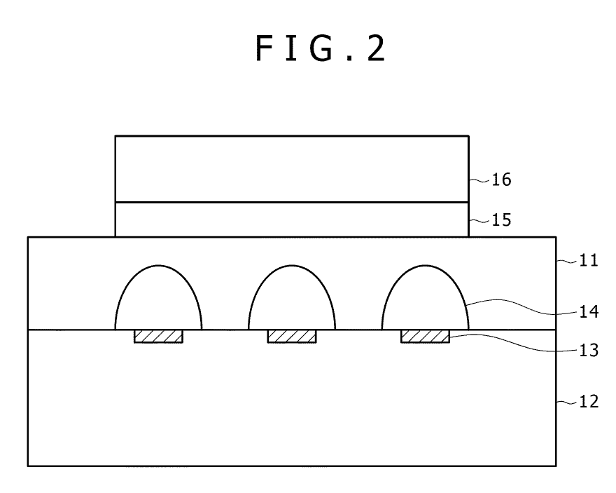

FIG. 2 is a conceptual diagram showing the reactor according to the embodiment of the present invention.

The reactor shown herein may be properly changed in size and layer structure according to objects. The shape and structure of the reactor 10 may be designed or modified within the scope of the present invention.

As shown in FIG. 2, the reactor 10 according to an embodiment of the present invention is composed of a well substrate 11, a heater substrate 12, a heating part (heater) 13, a reaction region 14 formed in the well substrate 11, a cooling part 15, and a radiator 16.

As explained above, the reactor 10 has the well substrate 11, which has a plurality of reaction regions 14, and the heating part 13, which heats the reaction region 14.

The cooling part 15 is a Peltier element which absorbs heat. Absorbed heat is released by the radiator 16.

The reaction regions 14 are intended for reactions under different conditions. Therefore, they permit a comprehensive analysis if reaction conditions are established individually for them.

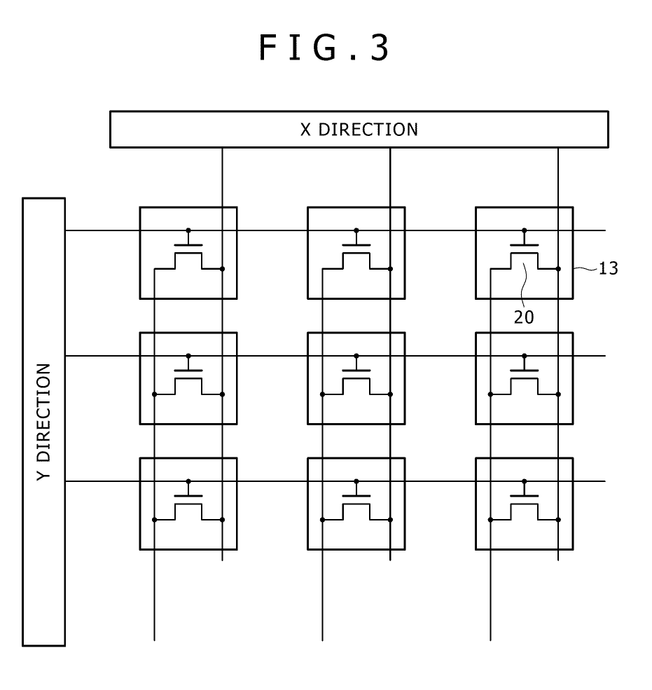

FIG. 3 is a schematic diagram showing the structure of the heating part in the reactor according to the embodiment of the present invention.

According to this embodiment, the reactor 10 has the reaction regions 14 arranged in a matrix pattern and each reaction region is provided with the heating part 13. All of the heating part 13 are arranged in a matrix pattern in the X and Y directions, as shown in FIG. 3.

This structure permits the semiconductor heat generating elements 20 to be controlled collectively.

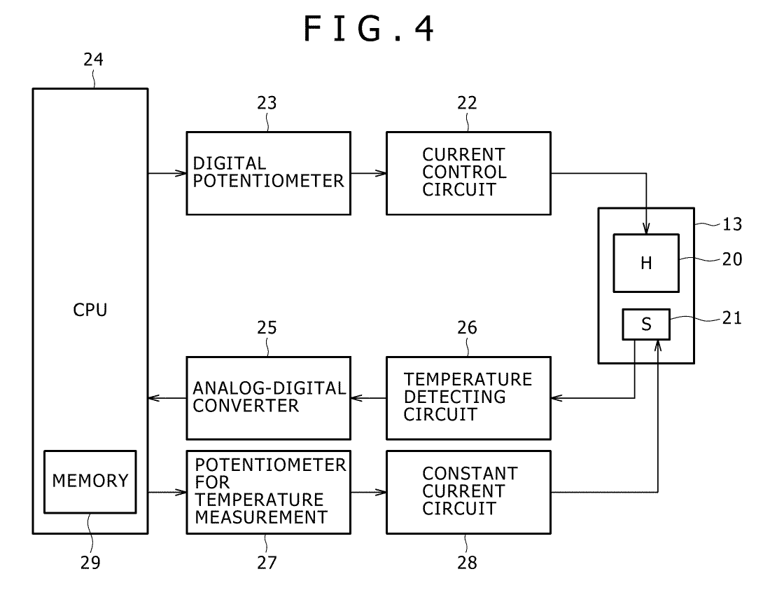

FIG. 4 is a schematic diagram showing the structure of the system to perform temperature control feedback for the control unit in the reactor according to the embodiment.

This system is intended to feed back the amount of heat generated by the semiconductor heat generating element 20 of the heating part (heater) 13 and also to feed back the temperature detected by the temperature detecting element 21. To this end, it is composed of a current control circuit 22, a digital potentiometer 23, a control unit (CPU) 24, an analog-digital converter (ADC) 25, and a temperature detecting circuit 26, a potentiometer for temperature measurement 27, a constant current circuit 28 and a memory 29.

The CPU 24 has the memory 29 inside, which stores parameters such as temperature information. It performs control in the same way even though it has the memory 29 outside.

The CPU 24 as the temperature control means performs processing for the temperature cycle which includes the first temperature holding control in denature treatment, the second temperature holding control in cooling from denature treatment to annealing treatment and also in annealing treatment, the first temperature rise control for the first heating from annealing treatment to extension treatment, the third temperature holding control in extension treatment, and the second temperature rise control for the second heating from extension treatment to denature treatment.

Also, the CPU 24 performs processing which includes the first temperature holding control in denature treatment, the second temperature holding control in cooling from denature treatment to annealing treatment and also in annealing treatment and extension treatment, and the temperature rise control in heating from extension treatment to denature treatment.

Incidentally, the temperature control means includes an analog-digital converter.

The CPU 24 also performs processing to detect temperature from the temperature detecting element 21, to calculate the amount of heater control, to control the heater 13, and to control the cooling element 15.

The CPU 24 also detects temperature from the temperature detecting element 21 by controlling current to be applied to the temperature detecting element 21 and converting the voltage of the temperature detecting element 21 by means of an analog-digital converter 25.

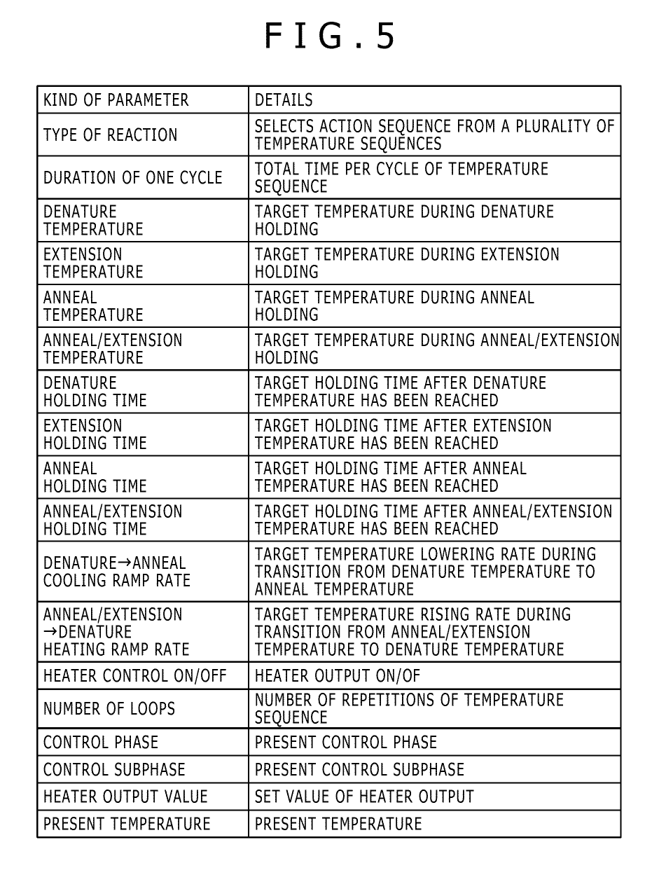

FIG. 5 shows one example of the control parameter used in the embodiment of the present invention.

The control parameters include the type of reaction, the duration of one cycle, the holding time, the anneal temperature, the heater control ON/OFF, the number of loops, the control phase, the heater output, and others.

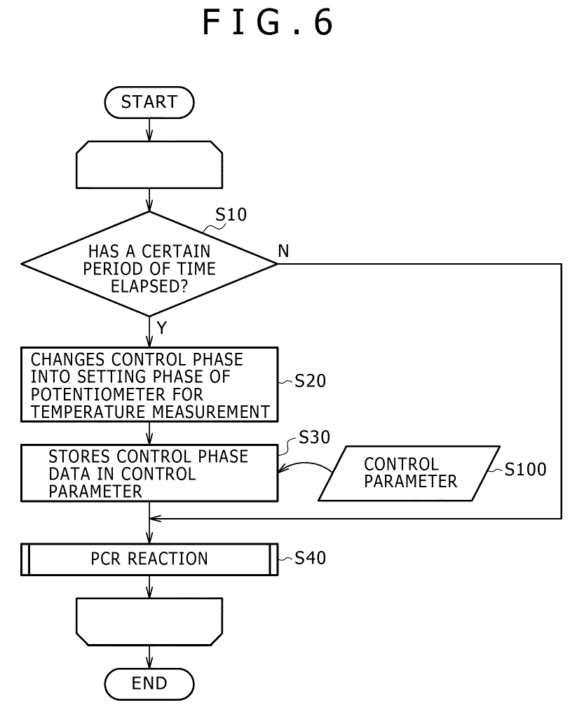

FIG. 6 is a flow chart illustrating the fundamental feedback control according to the embodiment.

The fundamental feedback control according to the embodiment is carried out as explained below with reference to FIG. 6.

The PCR process control in Step S40 is carried out periodically at fixed intervals. The lapse of the cycle time is determined in step S10. If it is determined that the cycle time has elapsed, the control phase parameter is changed into the potentiometer setting phase for temperature measurement in step S20.

Then, the control phase data is stored as the control parameter in step S30.

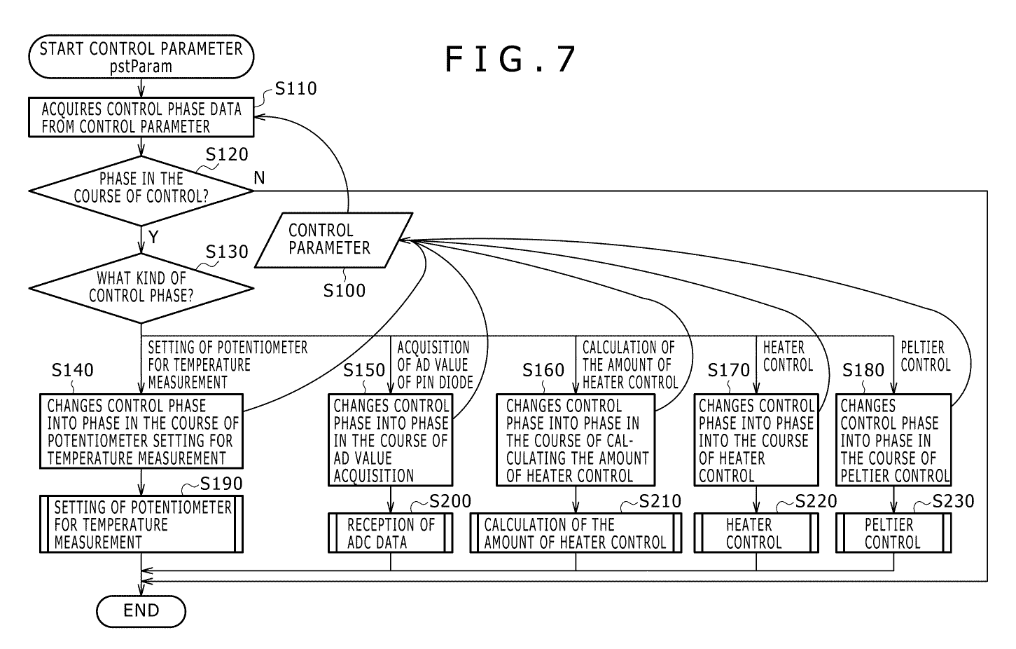

FIG. 7 is a flow chart illustrating how to control PCR process.

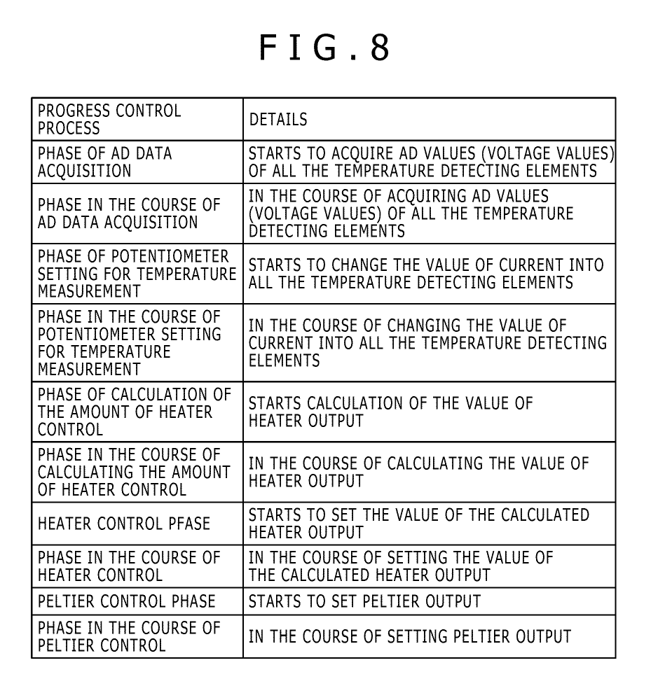

FIG. 8 is a list of controls (phases) in this embodiment.

The action of the PCR process will be explained below with reference to the flow chart shown in FIG. 7.

Each phase is processed as the control phase data is acquired from the control parameter S100 in Step S110.

Determination is made in Step S120 as to whether or not the phase is in the course of control.

If the phase is not in the course of control, the control phase is checked for its kind in Step S130.

In the case of potentiometer setting for temperature measurement, the control phase is changed into the phase in the course of potentiometer setting for temperature measurement in Step S140. And, the potentiometer for temperature measurement is set up in Step S190.

In the case of acquisition of AD value resulting from conversion from analog data of PIN diode into digital data, the control phase is changed into the phase in the course of AD value acquisition in Step S150. And, the AD value is received in Step S200.

In the case where the amount of heater control is calculated, the control phase is changed into the phase in the course of calculating the amount of heater control in Step S160. And, the amount of heater control is calculated in Step S210.

In the case of heater control, the control phase is changed into the phase in the course of heater control in Step S170. And, the heater control is performed in Step S220.

In the case of Peltier control, the control phase is changed into the phase in the course of Peltier control in Step S180. And, the Peltier control is performed in Step S230.

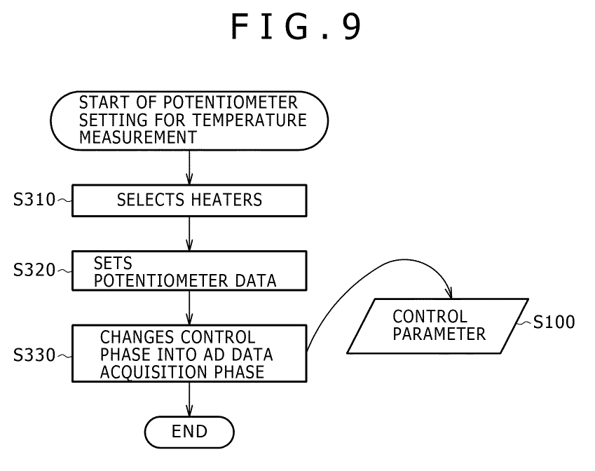

FIG. 9 is a flow chart illustrating how the phase of potentiometer setting for temperature measurement works.

The action of the phase of potentiometer setting for temperature measurement is explained below with reference to FIG. 9.

The heater to be controlled is selected in Step S310.

In Step S320, the potentiometer for temperature measurement is set up which is connected to the temperature detecting element in the same cell as the heater so that the temperature of the selected heater is measured.

After the setting of potentiometer for temperature measurement is completed, the control parameter is changed into the AD data acquisition phase in Step S330.

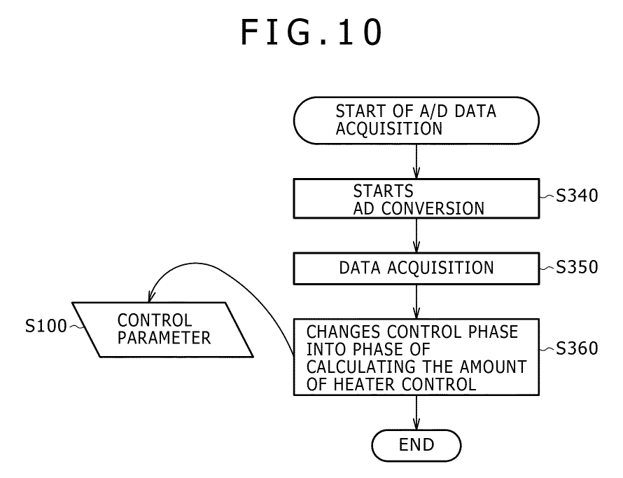

FIG. 10 is a flow chart illustrating how the AD data acquisition phase works.

The action of the AD data acquisition phase is explained below with reference to FIG. 10.

A command is sent to the analog-digital converter (ADC) to start analog-digital conversion in Step S340.

After AD conversion is completed, digital data is received from the analog-digital converter in Step S350.

After data acquisition, the control phase is changed into the heater control calculation phase in Step S360.

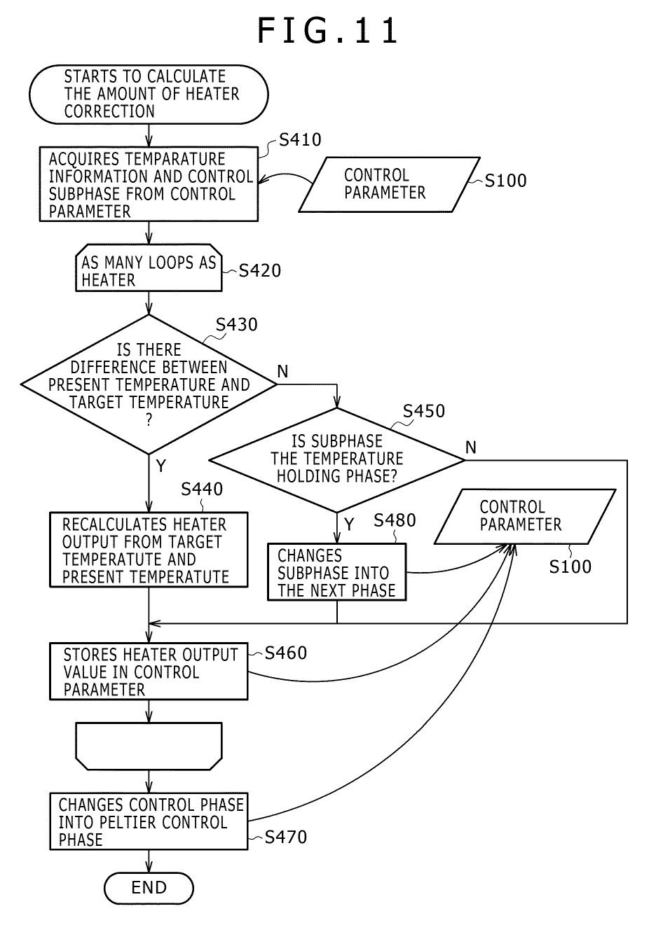

FIG. 11 is a flow chart illustrating how the phase of calculating the amount of heater control works.

The action of the phase of calculating the amount of heater control is explained below with reference to FIG. 11.

The temperature information and the control subphase are acquired from the control parameter in Step S410.

The control subphase represents the control step in PCR process when the heater output is calculated.

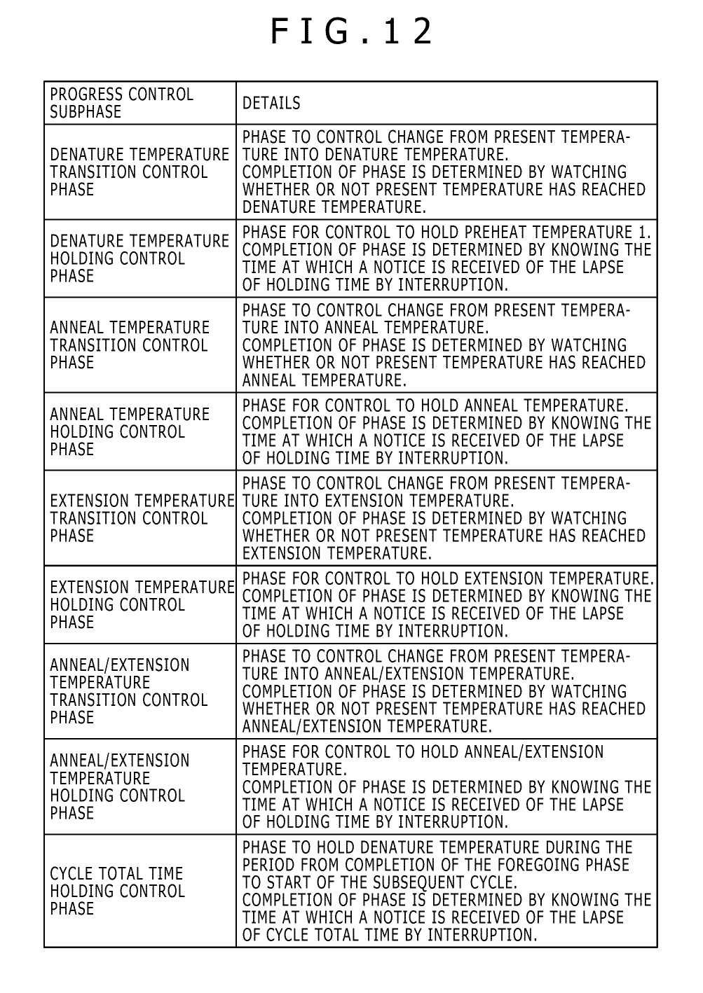

FIG. 12 is a list showing the control subphase in this embodiment.

Determination is made as to whether or not there exists difference between the present temperature and the target temperature in Step S430.

If there exists difference between the present temperature and the target temperature, the optimum heater output for the heater is calculated from the difference between the target temperature and the present temperature in Step S440.

If there exists no difference between the present temperature and the target temperature, determination is made as to whether or not the control subphase is the temperature holding phase in Step S450.

If the control subphase is the temperature holding phase, the control subphase is changed into the next phase and the result is stored in the control parameter in Step S480.

If the control subphase is not the temperature holding phase or if Step S440 or Step S450 has been completed, the heater output is stored in the control parameter in Step S460.

The Steps from S430 to S460 are repeated as many times as the number of heaters to be controlled in Step S420.

The control phase is changed into the Peltier control phase in Step S470.

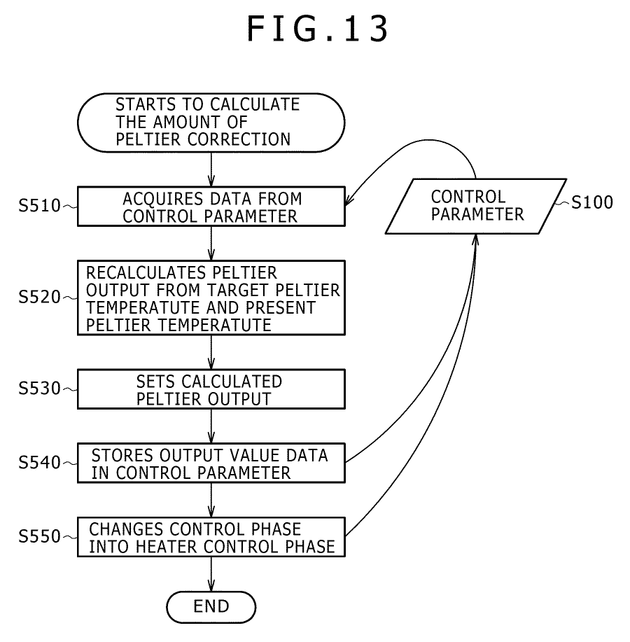

FIG. 13 is a flow chart illustrating how the Peltier control phase works.

The action of the Peltier control phase is explained below with reference to the flow chart shown in FIG. 13.

The Peltier set temperature and the present Peltier temperature are acquired from the control parameter in Step S510.

The Peltier output is calculated from the target Peltier temperature and the present Peltier temperature in Step S520.

The optimum Peltier temperature is set from the Peltier set temperature and the present Peltier temperature (both acquired in Steps S510 and S520) in Step S530.

The Peltier set temperature is stored in the control parameter in Step S540.

The control phase is changed into the heater control phase in Step S550.

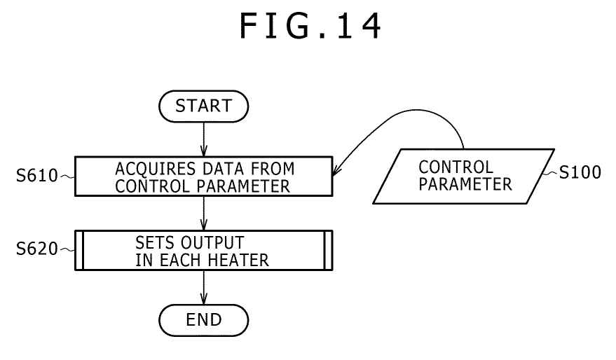

FIG. 14 is a flow chart illustrating how the heater control phase works.

The action of the heater control phase is explained below with reference to the flow chart shown in FIG. 14.

The heater set value is acquired from the control parameter in Step S610.

The set value is sent to each heater in Step S620.

The specific method of output is explained with reference to FIG. 4. The CPU 24 supplies the digital potentiometer 23 with digital values. The heating element 13 is kept at a controlled temperature by the digital potentiometer 23 and the current control circuit 22.

As mentioned above, this embodiment allows more accurate temperature control through temperature control feedback based on the temperature information detected by means of the temperature detecting element 21.

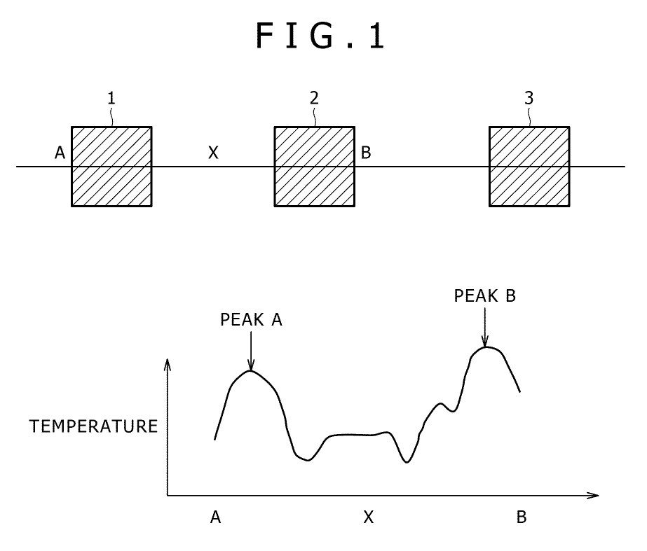

In the event of heat diffusion as shown in FIG. 1, the temperature detecting element 21 observes heat generation exceeding the set value and rapidly changes the set value of the heater 20. In addition, the fact that the heater 20 is provided individually with the temperature detecting element 21 for control to be performed independently and individually permits all the heaters 20 to be controlled accurately regardless of their position in the matrix.

Explained below is the heat control matrix device to which the above-mentioned heater control method can be applied and which can be applied to the reactor 10 for PCR process.

The reactor for PCR process includes, for example, the real-time PCR apparatus to detect gene expression.

The PCR apparatus is basically provided with the semiconductor heat generating part (heater) 20, the temperature detecting part (element) 21, and the fluorescence detector.

The PCR apparatus may be constructed such that the reaction signal is received by a separate functioning part which is formed above or under the TFT substrate serving as the heating part. In this case the heater matrix should preferably be formed on a comparatively large transparent insulating substrate (such as glass) which will not prevent detection of fluorescence for reaction signals.

To this end, it is desirable to use thin film transistors (TFT for short hereinafter) as the semiconductor elements from the standpoint of production cost and manufacturing process.

It is known that, however, TFT is more liable to variation in manufacturing process and change with time than single-crystal semiconductor elements.

To be more specific, the heater in the PCR apparatus should preferably be formed by low-temperature polysilicon process that forms TFT (suitable for current drive) on a large glass substrate. This process usually consists of coating a glass substrate with an amorphous silicon film and crystallizing by laser annealing for protecting the substrate from thermal deformation).

The disadvantage of this process is that a large glass substrate involves difficulties in uniform irradiation with laser energy and hence inevitably varies in the state of crystallization of polysilicon from one place to another. As the result, TFTs formed on the same substrate may vary in threshold value (Vth) by more than hundreds of mV or even more than 1 V. With such TFTs, it is difficult to construct a highly accurate and reliable PCR reactor by the existing technology.

In order to overcome this difficulty, the following embodiment is proposed in which the PCR apparatus with TFTs formed on a transparent insulating substrate achieves highly accurate temperature control with the help of a heat control matrix device.

To be concrete, the embodiment mentioned below is designed to achieve highly accurate temperature control by constituting heater units from TFTs with current copy circuit or current mirror circuit. Moreover, it is also designed to achieve a highly accurate comprehensive analysis by performing feedback with the help of a PIN diode as a sensor and by detecting fluorescence as amplification reaction signals with the help of parallel PIN diode.

The heat control matrix device pertaining to this embodiment may also be used as the heating part 13, temperature detecting part, or fluorescence detecting part of PCR 1 mentioned above.

The embodiment for the heat control matrix device covers the following ones which will be described below one by one.
Heater matrix device that can be used as the heating part (or heat generating part) capable of controlling the amount of heat generation.
Temperature detecting matrix device that can be used as the temperature detecting part.
Fluorescence detecting matrix device that can be used as the fluorescence detecting part.
Temperature fluorescence detecting matrix device that functions as both the temperature detecting matrix device and the fluorescence detecting matrix device.
Heater temperature detecting matrix device that functions as both the heater matrix device and the temperature detecting matrix device.
Heater temperature fluorescence detecting matrix device that functions as both the heater matrix device and the temperature fluorescence detecting matrix device.

The heater matrix device will be explained first.

### <Heater Matrix Device>

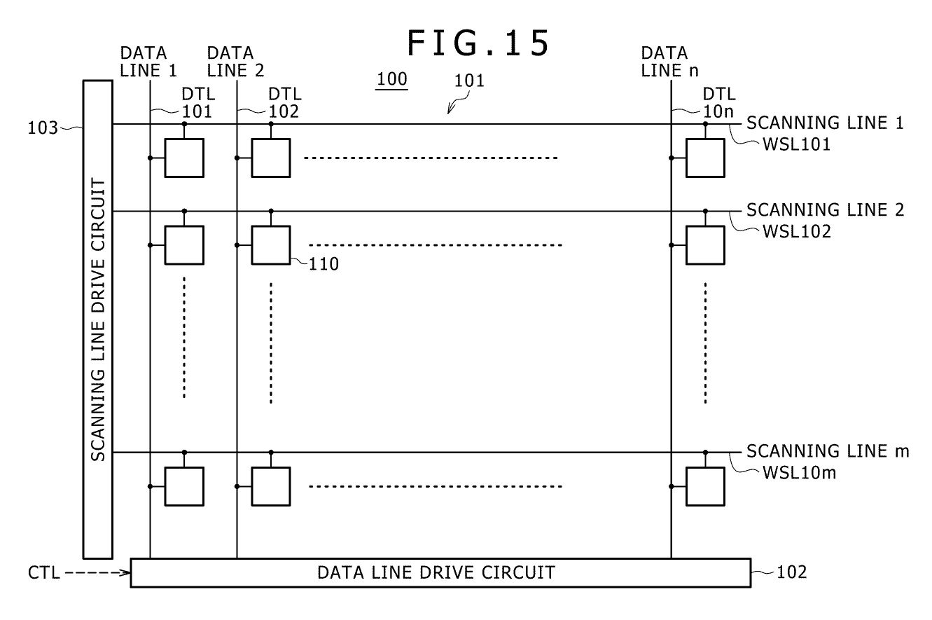

FIG. 15 is a schematic diagram showing the structure of the heater matrix device according to the embodiment of the present invention.

The heater matrix device 100 shown in FIG. 15 consists of the cell array 101 with heater units 110 arranged in an m x n matrix pattern, the data line driving circuit (DTDRV) 102, the scanning line driving circuit (WSDRV) 103, the data lines DTL101 . . . DTL10n which give the heater units 110 the information about the amount of heat generation, and the scanning lines WSL101 . . . WSL10m which select the heater units 110, write the information about the amount of heat generation, and supply current in response to the written information about the amount of heat generation.

The data line driving circuit 102 applies signal current to each of the data lines DTL101 . . . DTL10n in synchronism with the driving timing of the scanning lines WSL101 . . . WSL10m of the scanning line driving circuit 103, thereby writing the information about the amount of heat generation to the heater unit 110 as the heating part for each row.

The scanning line driving circuit 103 sequentially selects the scanning lines WSL101 . . . WSL10m for pulse driving. The scanning line driving circuit 103 drives the scanning lines WSL101 . . . WSL10m to control the timing at which the heater unit 110 acquires the information about the amount of heat generation.

The scanning line driving circuit 103 writes the information about the amount of heat generation to the heater unit 110 and then unselects the scanning lines WSL101 . . . WSL10m, thereby continuing to supply each heat generating part (heater unit) with the driving current of the same magnitude as the signal current.

In this way it supplies each heater unit 110 with as much current as necessary to generate heat in a desired amount.

Incidentally, the data line driving circuit 102 transfers signal current, which is the information about the amount of heat generation in response to the control signal CTL supplied from the temperature detecting and controlling system (not shown), to each data line DTL101 DTL10n, thereby controlling the amount of heat generated by each heater unit 110.

In other words, the amount of heat generated by the heater unit 110 is controlled by the information about the amount of heat generation which has been written.

The heater unit 110 is constructed as explained in the following.

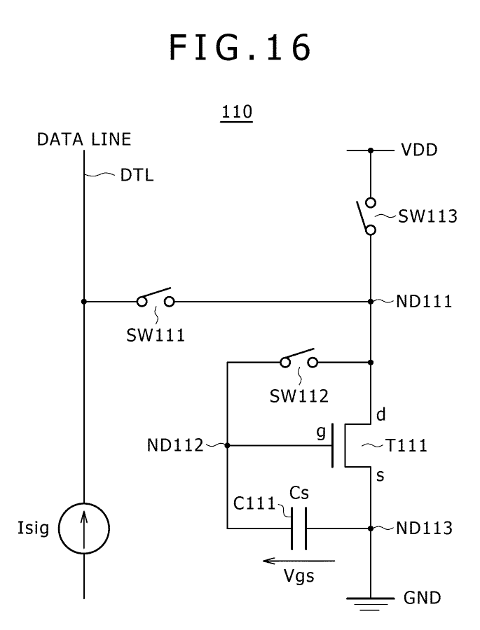

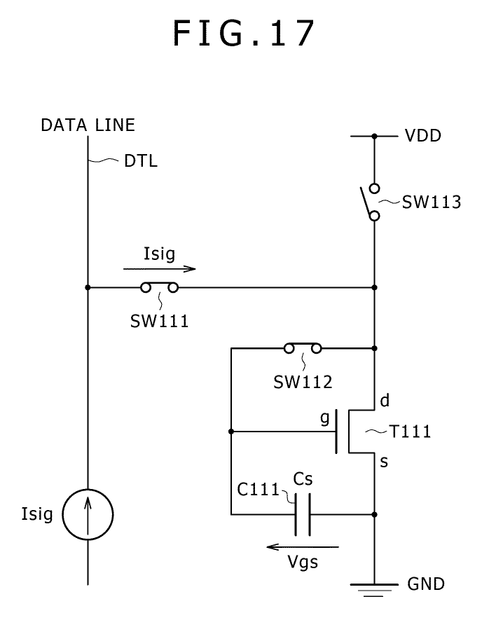

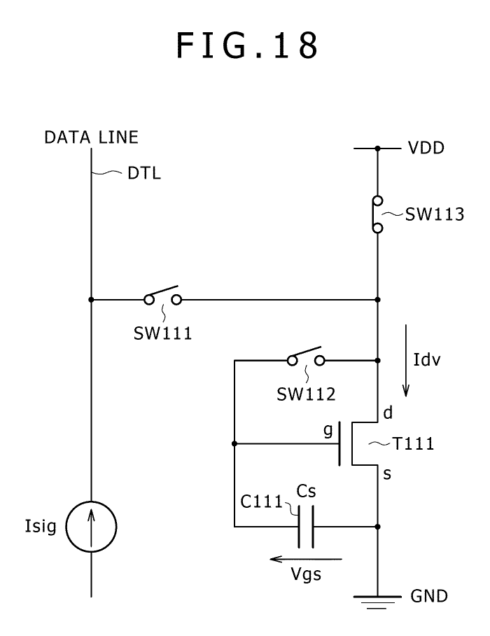

FIG. 16 is a circuit diagram showing a first example of the structure of the heater unit in the heater matrix device according to the embodiment of the present invention. FIG. 17 is a circuit diagram showing one activated state of the circuit shown in FIG. 16. FIG. 18 is a circuit diagram showing another activated state of the circuit shown in FIG. 16.

The heater unit 110 shown in FIG. 16 consists of the transistor T111 which is an n-channel insulated gate transistor, the switches SW111, SW112, and SW113, the capacitor C111, and the nodes ND111, ND112, and ND113. Incidentally, symbols g, d, and s in FIG. 9 represent gate, drain, and source, respectively, and symbol Cs denotes the capacity of the capacitor C111.

The heater unit 110 is constructed such that the transistor 111 which functions as a driving transistor has its drain d, gate g, and source s connected respectively to the nodes ND111, ND112, and ND113. The node ND113 is connected to the ground potential GND.

The switch SW111 is connected to the data line DTL which transmits signal current I_{sig} and the node ND113. The switch SW112 is connected to the node ND111 and the node ND112. The switch SW113 is connected to the node ND111 and the source potential VDD.

The capacitor C111 is connected to the node ND112 through its first electrode and the node ND113 (or ground potential GND) through its second electrode.

In the heater unit 110, the switches SW111 and SW112 turn on and off in phase in response to the level of the scanning lines WSL101 . . . WSL10m.

The switch SW113 turn on and off complimentarily to the switches SW111 and SW112 in response to the level of the scanning lines WSL101 . . . WSL10m.

Of these constituents, the switches SW111 and SW112 receive the information about the amount of heat generation which is given to the data line DTL when the scanning line WSL is selected.

The capacitor C111 holds the information about the amount of heat generation even after the scanning line has been unselected.

And, the transistor T111 and the switch SW113 allow current to flow according to the written information about the amount of heat generation, and they function as the driver to generate heat in response to the current.

In the heater unit 110, the driving current flows from the source potential VDD to the ground potential GND through the transistor T111 and the switch SW113.

The resistance of the transistor T111 and the switch SW113 generates Joule heat to be used as the heat source.

Incidentally, the transistor T111 is not limited to n-channel one; it may be replaced by p-channel one.

In this embodiment, the information about the amount of heat generation which is transmitted from the data line DTL is signal current I_{sig}. Therefore, it is desirable to construct a circuit which controls heat by converting this signal current into signal voltage. The action of the circuit shown in FIG. 9 will be described with reference to FIGS. 17 and 18.

FIG. 17 shows the action of writing to the heater unit 110 the information about the amount of heat generation in the form of current level (or signal current). During this writing action, the switches SW111 and SW112 are on and the switch SW113 is off.

The transistor T111 permits the signal current I_{sig} to flow, with the drain d and the gate g shorted by the switch SW2. See FIG. 17.

As the result, the signal voltage V_{gs} occurs between the gate and the source in response to the value of the signal current I_{sig}.

In the case where the transistor T111 is that of enhancement mode (or the threshold value Vₜₕ>0), it works in the saturation region. Thus the signal current I_{sig} and the signal voltage V_{gs} are related to each other by the following well-known equation.

### [Equation 1]

I_{sig}=μ·Cₒₓ·W/L/2·(V_{gs}−Vₜₕ² (1)

In the equation above, μ denotes the carrier mobility, Cₒₓ denotes the gate capacity per unit area, W denotes the channel width, and L denotes the channel length.

When the circuit becomes stable, the switch SW112 turns off so that the gate-source voltage V_{sg} is stored in the capacitor C111. Then the switch SW111 turns off to complete the signal writing action.

Then, the switch SW113 turns on at any timing as shown in FIG. 18, so that current flows from the source voltage VDD to the ground potential GND. At this time, the driving current I_{drv} flowing through the transistor T111 is represented by the equation (2) below irrespective of the source-drain voltage V_{ds} if source voltage VDD is set sufficiently high and the resistance of the switch SW113 is set sufficiently low so that the transistor T111 works in the saturation region. And the driving current I_{drv} coincides with the signal current I_{sig} mentioned above.

### [Equation 2]

I_{drv}=μ·Cₒₓ·W/L/2·(V_{gs}−Vₜₕ)² (2)

In general, the parameters that appear in the right side of the equations (1) and (2) above vary from one substrate to another or vary from one position to another in the same substrate. However, driving as shown in FIGS. 17 and 18 makes the signal current I_{sig} to coincide with the driving current I_{drv} irrespective of the values of the individual parameters.

Since the signal current I_{sig} mentioned above can be generated accurately by the control circuit outside the heater matrix device, Joule heat generated by the heater unit (shown in FIG. 16) has an accurate value determined by VDD×I_{sig} (or the product of the source voltage VDD and the signal current I_{sig}) without being affected by variation in transistor characteristics.

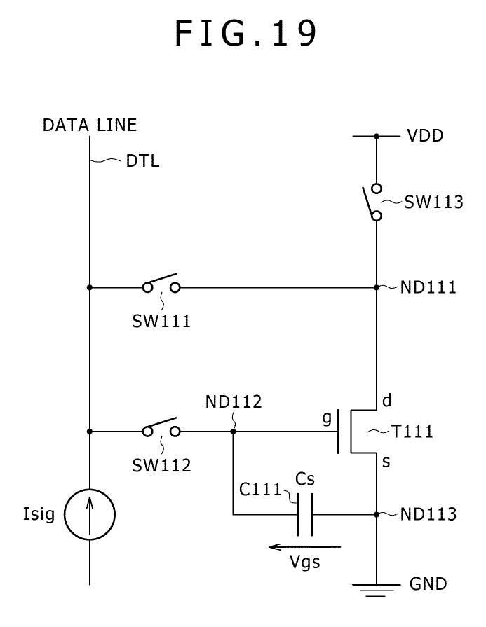

FIG. 19 is a circuit diagram showing a modified example of the circuit shown in FIG. 16.

The circuit shown in FIG. 19 differs from that shown in FIG. 16 in the connection of the switch SW112. To be specific, the switch SW112 is placed between the data line DTL and the node ND112 instead of being placed between the node ND111 and the node ND112.

The circuit shown in FIG. 19 is equivalent in its action to the circuit shown in FIG. 16; the difference is that the node ND112 is connected to the data line DTL through the switch SW111 and the node ND111 in FIG. 16.

The circuit shown in FIG. 119 works in the same way as that shown in FIG. 16. That is, the switches 111 and 112 turn on and the switch SW113 turns off at the time of signal writing. And, the switches SW111 and SW112 turn off and the switch SW113 turns on at the time of heat generation.

The circuit shown in FIG. 119 functions in the same way as the circuit shown in FIG. 16.

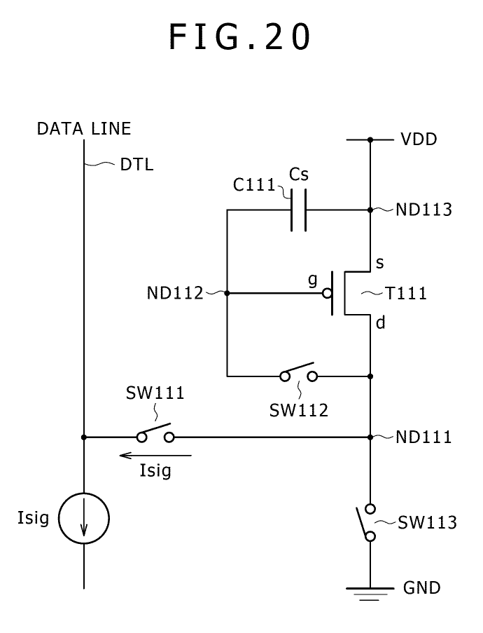

FIG. 20 is a circuit diagram showing another modified example of the circuit shown in FIG. 16.

The circuit shown in FIG. 20 differs from that shown in FIG. 16 in that the transistor T111 is a p-channel transistor and the direction of current is reversed.

In the case of the circuit shown in FIG. 20, the source s of the transistor T111 is connected to the source potential (node ND113), the drain d of the transistor T111 is connected to the node ND111, and the switch SW113 is connected to the intermediate point between the node ND111 and the ground potential GND.

The circuit shown in FIG. 20 is in principle common to that shown in FIG. 16 and both function in the same way.

According to the embodiment of the present invention, it is desirable to employ a p-channel insulation gate transistor (PMOS) for the low-temperature polysilicon thin film transistor (TFT) because of its stable characteristics.

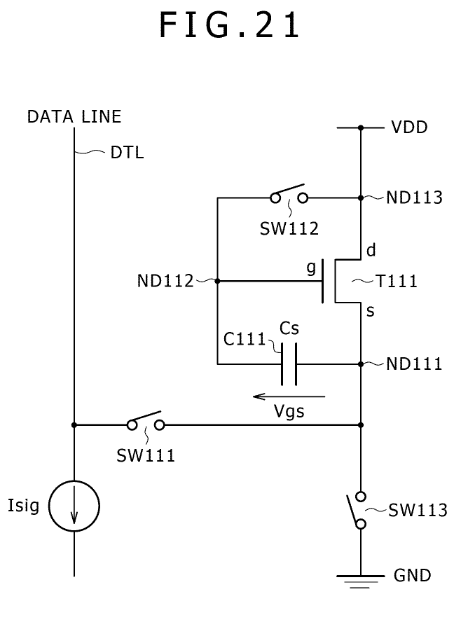

FIG. 21 is a circuit diagram showing further another modified example of the circuit shown in FIG. 16.

The circuit shown in FIG. 21 is identical with that shown in FIG. 16 in the way the switches SW111, SW112, and SW113 are controlled but it is so designed as to draw the signal current I_{sig} from the source of the transistor T111.

In the case of the circuit shown in FIG. 21, the transistor T111 is an n-channel transistor and the drain d of the transistor T111 is connected to the source potential (VDD), the source s of the transistor T111 is connected to the node ND111, and the switch SW113 is connected to the intermediate point between the node ND111 and the ground potential GND.

The circuit shown in FIG. 21 works in the same way as that shown in FIG. 16 in that it permits the signal current I_{sig} to flow while the gate and the drain are shorted to each other and the resulting gate-source voltage V_{gs} is stored in the capacitor C111. Both function in the same way.

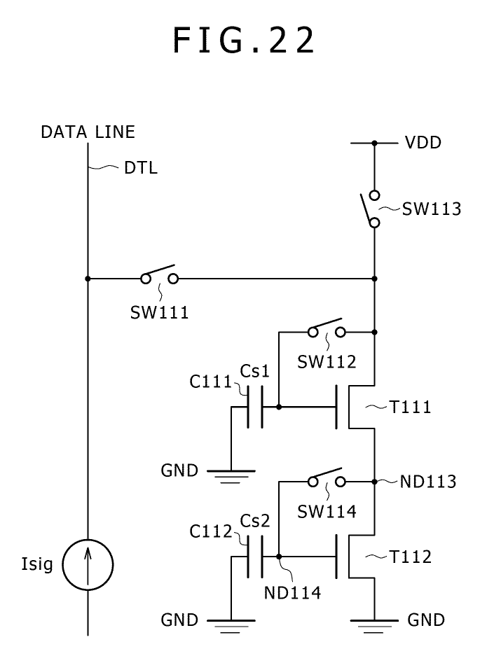

FIG. 22 is a circuit diagram showing further another modified example of the circuit shown in FIG. 16.

The circuit shown in FIG. 22 differs from that shown in FIG. 16 in that it additionally has the transistor T112, the switch SW114, and the capacitor 112. The switch SW114 is controlled in the same way as the switch SW112.

The transistor T112 has its gate connected to the node ND114, its drain connected to the node ND113, and its source connected to the ground potential GND. The switch SW114 is connected to the intermediate point between the node ND113 and the node ND114. The capacitor C112 has its first electrode connected to the node ND114 and its second electrode connected to the ground potential GND.

This circuit works in the following way.

In the circuit shown in FIG. 16, the signal current I_{sig} is given by the equation (1), the drive current I_{drv} is given by the equation (2), and the signal current I_{sig} coincides with the drive current I_{drv}, as mentioned above. This fact accords with the principle that the current flowing through a MOS (metal oxide semiconductor) transistor depends only on the gate-source voltage V_{gs} irrespective of the drain-source voltage V_{ds} for action in the saturation region.

However, in a practical transistor, an increase in the drain-source voltage V_{ds} usually results in a slight increase in the drain-source current I_{ds}. Probably, this is due to the back gate effect (the potential of the drain affects the conduction state of the channel) and the short channel effect (the depletion layer at the end of the drain extends to the source side to shorten the effective channel length L).

This will be illustrated with reference to the circuit shown in FIG. 16. In the case where a comparatively small signal current I_{sig} is written, the gate-source voltage V_{gs} that arises according to the equation (1) is a comparatively small value and the drain-source voltage V_{ds} is a small value equal to the gate-source voltage V_{gs}.

On the other hand, at the time of driving, the drive current I_{drv} is small and hence the voltage drive across the switch SW113 is small, and the drain-source voltage V_{ds} of the transistor T111 becomes a larger value than that at the time of writing. Thus, usually the drain-source voltage V_{ds} at the time of writing is not equal to that at the time of driving. Consequently, the signal current I_{sig} and the drive current I_{drv} do not exactly coincide with each other. This may be a reason why the desired amount of heat generation is not obtained.

By contrast, the circuit shown in FIG. 22 functions in the following manner.

As in the circuit shown in FIG. 16, the drain-source voltage V_{ds} of the transistor T111 at the time of writing usually varies from that at the time of driving.

However, when the drain-source voltage V_{ds} is large at the time of driving, the drive current I_{drv} becomes larger than the signal current I_{sig}, however, if the transistor T112 is working in its saturation state (or working close to the constant current source), its differential resistance takes on a very large value.

Thus, with a slight increase in the drive current I_{drv}, the source potential of the transistor T111 greatly increases. This reduces the gate-source voltage V_{gs} of the transistor T111 and also decreases the drive current I_{drv}.

As the result, the drive current I_{drv} does not increase so much relative to the signal current I_{sig}, and coincidence between the drive current I_{drv} and signal current I_{sig} becomes better than that in the case shown in FIG. 16.

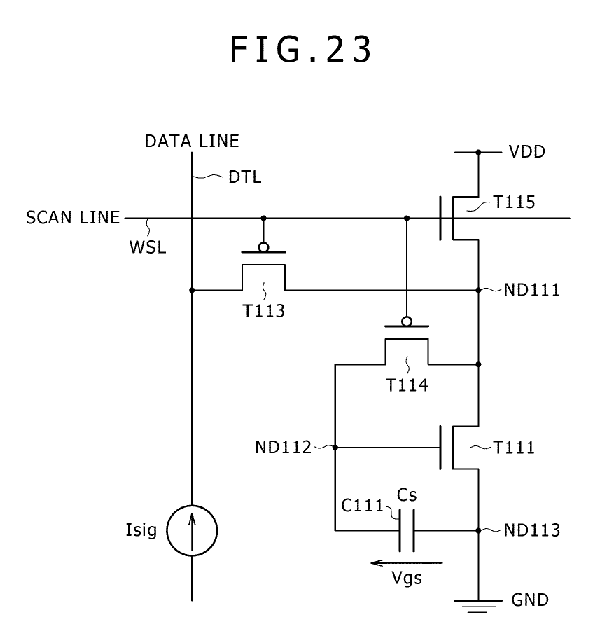

FIG. 23 is a circuit diagram showing a typical example of the circuit shown in FIG. 23.

The circuit shown in FIG. 23 is composed of the p-channel transistor T113 (which functions as the switch SW111), the p-channel transistor T114 (which functions as the switch SW112), and the n-channel transistor T115 (which functions as the switch SW113).

These three transistors T113, T114, and T115 have their gates commonly connected to the scanning line WSL. When the scanning line WSL is at a low level, signal writing is accomplished, and when it is at a high level, drive action is performed.

As mentioned later, the present invention may be modified such that the transistors T113, T114, and T115 do not have their gates commonly connected to the scanning line WSL. However, the circuit shown in FIG. 22 is desirable because of its simple structure.

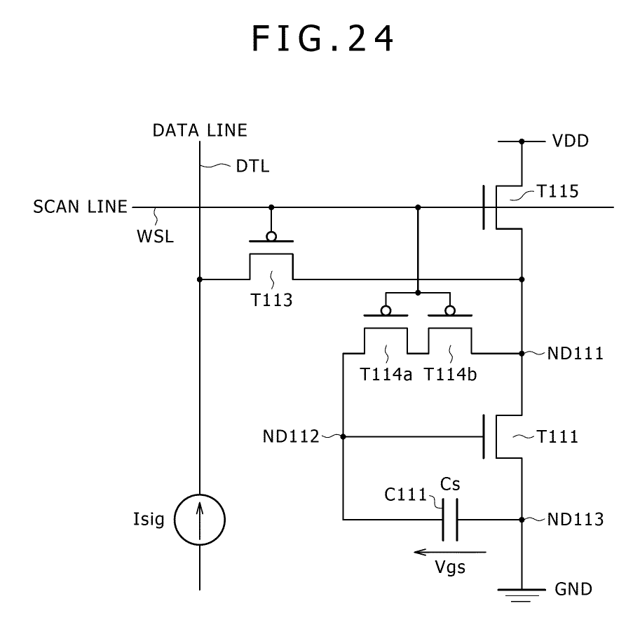

FIG. 24 is a circuit diagram showing a modified example of the circuit shown in FIG. 23.

The circuit shown in FIG. 24 differs from that shown in FIG. 23 in that it has the transistors T114a and T114b.

TFTs are usually liable to become defective in the manufacturing process. For example, there is the possibility that the switch transistor permits a minute leakage current to flow when it is off.

The circuit shown in FIG. 23 works in such a way that when a leakage current occurs in the transistor T114, the leakage current changes the voltage held in the capacitor C111. This leads to a situation in which adequate heat generation cannot be maintained.

By contrast, the circuit shown in FIG. 24, which has the two transistors T114a and T114b connected in series in place of the one transistor T114 used in the circuit shown in FIG. 23, is able to suppress leakage current as a whole even though one of the two transistors is defective.

By the same token, the transistor T114 may be replaced by three or more transistors connected in series or each of the transistors T113 and T115 may be replaced by more than one transistor connected in series.

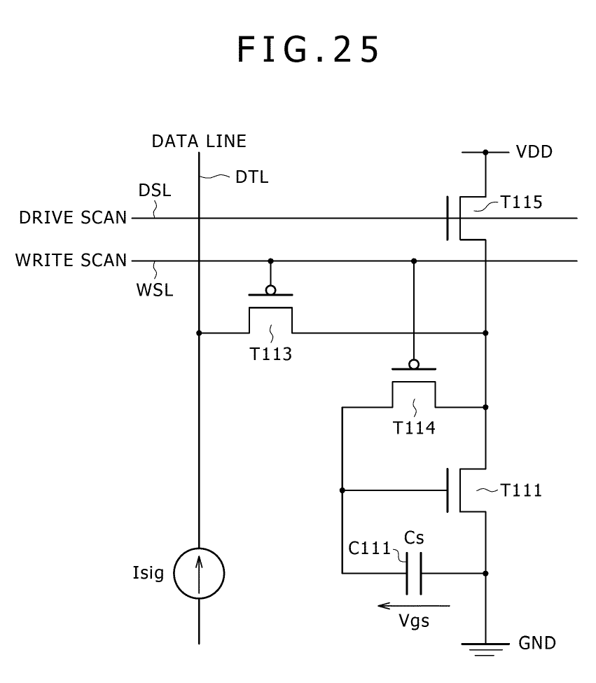

FIG. 25 is a circuit diagram showing another modified example of the circuit shown in FIG. 16.

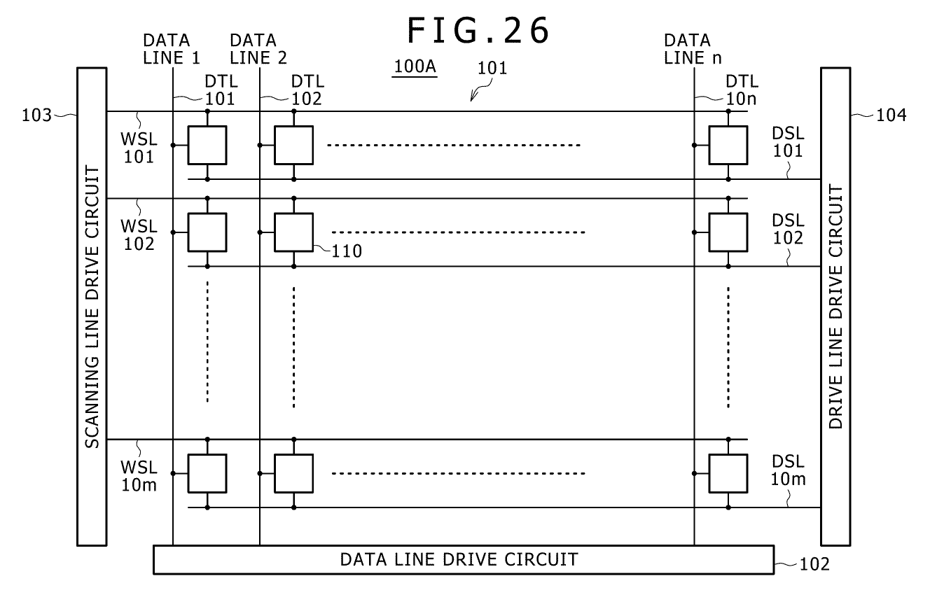

FIG. 26 is a schematic diagram showing the structure of the heater matrix device having the heater unit shown in FIG. 25.

The circuit shown in FIG. 25 is constructed such that the transistor T115 is controlled independently of the transistors T113 and T114.

The heater matrix device 100A shown in FIG. 26 differs from that shown in FIG. 15 in that it additionally has the drive line driving circuit 104 and the drive scanning lines DSL101 . . . DSL10m which drive the transistor T115.

In this case, at the time of signal writing, the write scanning lines SWL101 . . . SWL10m and the drive scanning lines DSL101 . . . DSL10m are kept low.

After writing has been completed (or after the write scanning lines have been made high), the drive scanning lines DSL101 . . . DSL10m are made high at arbitrary timing, so that heat generation is activated.

Conversely, as the drive scanning lines DSL101 . . . DSL10m are made low, heat generation can be suspended easily; this is desirable when it is necessary to lower temperature rapidly. This leads to capability of adjusting the duration of heat generation. That is, the device can produce a very small amount of heat very accurately even when the signal current source cannot generate a small current accurately.

Incidentally, in the case where it is desirable to avoid intermittent heating due to the foregoing action, the steps for heat generation and suspension of heat generation should be repeated several times in the period from the writing of the information about the amount of heat generation to the next writing of the information about the amount of heat generation. This ensures temporal stability of heat generation.

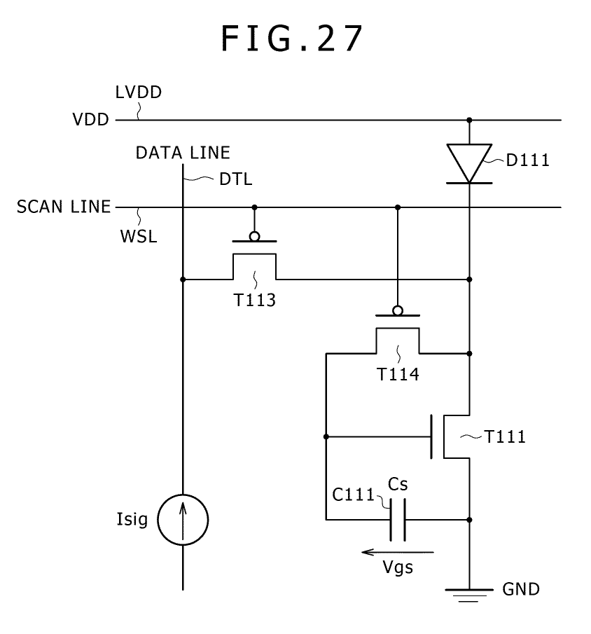

FIG. 27 is a circuit diagram showing another modified example of the circuit shown in FIG. 16.

In FIG. 27, the supply potential line LVDD is parallel to the scanning line WSL and the diode D111 is equivalent to the switch SW113 shown in FIG. 16.

At the time of signal writing, the source voltage VDD is brought to a low level to turn off the diode D111, and at the time of driving, the source voltage VDD is brought to a high level to turn on the diode D111. In this way the diode D111 functions as a switch Thus, the circuit shown in FIG. 27 functions in the same way as the circuit shown in FIG. 25.

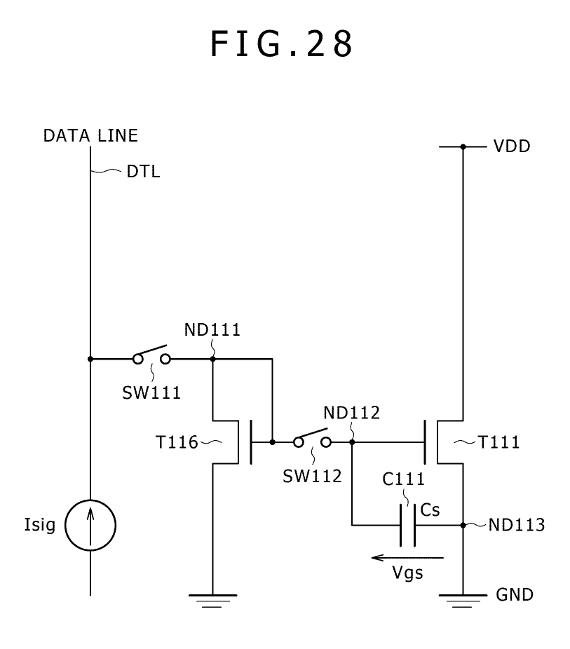

FIG. 28 is a circuit diagram showing further another modified example of the circuit shown in FIG. 16.

The circuit shown in FIG. 28 differs from that shown in FIG. 16 in that it has the transistor T116 to convert the signal current I_{sig} into a voltage and the transistor T111 to permit current to flow for heat generation.

The transistor T116 has its drain and gate connected to each other, with the connecting point connected to the nodes ND111 and ND112, and the transistor T116 has its source connected to the ground potential GND.

At the time of signal writing, the switches SW111 and SW112 becomes on to supply the signal current I_{sig} to the transistor T116. In this situation, the following equation (3) holds.

### [Equation 3]

I_{sig}=μ·Cₒₓ−W1/L/2·(V_{gs}−Vₜₕ)² (3)

The parameters in Equation (3) are defined as in Equation (1). The transistor T116 has a channel width of W₁. At the time of driving, the switches SW111 and SW112 tun off.

On the other hand, the capacitor C111 holds the gate-source voltage V_{gs} due to writing, so that the drive current I_{drv} flowing through the transistor T111 accords with the following equation (4).

### [Equation 4]

I_{drv}=μ·Cₒₓ·W2/L/2·(V_{gs}−Vₜₕ)² (4)

Since the transistor T111 has channel width of W₂ and the transistors T116 and T111 are formed in a minute heating part, Cₒₓ and Vₜₕ, which are the parameters of the Transistors T116 and T111, are considered to be equal to each other. Moreover, the channel length L can be designed to be identical for these transistors. As the result, the equations (3) and (4) yield the following equation (5).

I_{drv}/I_{sig}=W₂/W₁ (5)

In general, the parameters in the right side of the equations (3) and (4) above vary from one substrate to another or vary from one position to another in the same substrate. It is known that these parameters have nothing to do with the ratio between the signal current I_{sig} to the drive current I_{drv} which coincides with the ratio between the channel width of the transistor T111 and the channel width of the transistor T116.

This circuit differs from that shown in FIG. 16 in that it makes it possible to arbitrarily adjust the ratio between the signal current I_{sig} and the drive current I_{drv}. If it is desirable to generate a very small amount of heat but the external circuit cannot generate a very small amount of current, then this problem is solved by designing the channel width such that the right side of the equation (5) takes on a small value. Conversely, it is also easy to design such that a very small signal current I_{sig} can control a large drive current I_{drv}.

The foregoing is a description of the heater matrix device.

The following is a description of the temperature detecting matrix device.

### <Temperature Detecting Matrix Device>

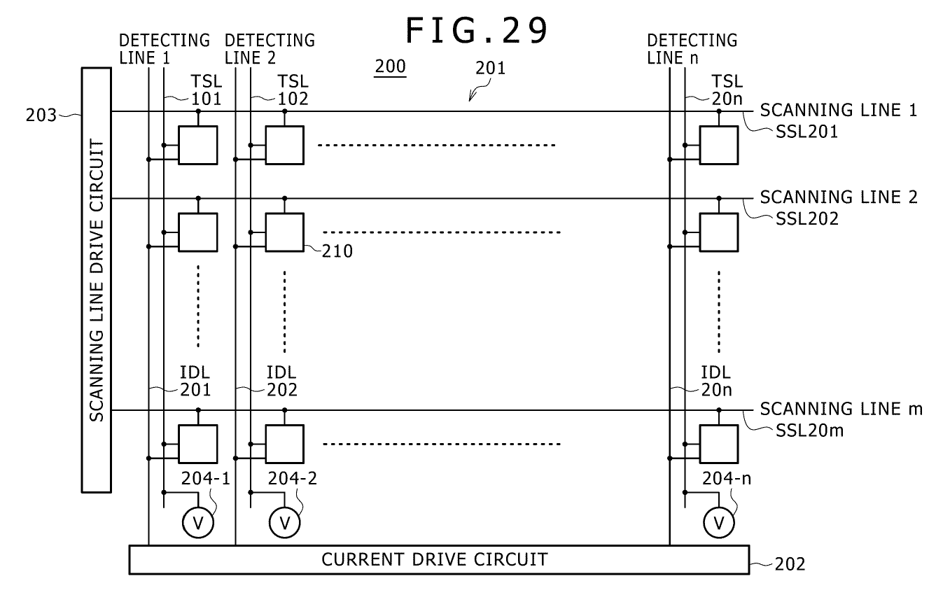

FIG. 29 is a schematic diagram showing the structure of the temperature detecting matrix device according to the embodiment of the present invention.

The temperature detecting matrix device 200 shown in FIG. 29 consists of the cell array 201 of temperature detecting units 210 arranged in an m x n matrix pattern, the current driving circuit (IDRV) 202, the scanning line driving circuit (WSDRV) 203, the voltage detecting lines (V) 204-1 . . . 204-n, the current driving lines IDL201 . . . IDL20m, the temperature sense lines TSL201 . . . TSL20m, and the scanning liens SSL201 . . . SSL20m, which select the temperature detecting units 210 and send the detected signals from the temperature detecting unit 210 to the temperature sense lines TSL201 . . . TSL20m.

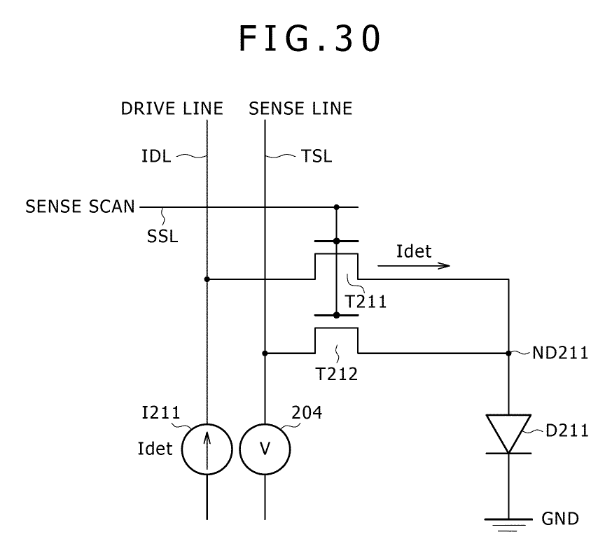

FIG. 30 is a circuit diagram showing the structure of the temperature detecting unit according to the embodiment of the present invention.

The temperature detecting unit 210 shown in FIG. 30 has the PIN diode D211, the n-channel transistors T211 and T212 which function as switches, and the node ND211.

The PIN diode 211 has its anode connected to the node ND211 and its cathode connected to the ground potential GND.

The transistor T211 has its source and drain connected to the node ND211 and the current driving line IDL, respectively. The transistor T212 has its source and drain connected to the node ND111 and the temperature detecting line TSL.

And, the transistors T211 and T212 have their gates connected in common to the scanning line SSL.

The transistors T211 and T212 turned on when the scanning line SSL is at a high level and are turned off when the scanning line SSL is at a low level.

The temperature detecting unit 210 functions in the following manner.

It is connected to the current source I211 that supplies current I_{det} to the current driving line IDL, so that the forward current I_{det} flows to the PIN diode D211 from the current source I211 connected to the current driving line IDL when the scanning line SSL is at a high level.

At the same time, the voltage detector 204 is connected to the temperature sense line TSL, so that the forward voltage that occurs in the PIN diode D211 is detected. The voltage detector 204 may be an analog-digital converter.

The temperature detecting unit 210 detects temperature as the PIN diode D211 senses dark current. The thus detected temperature is referenced to control the amount of heat generation by each heater unit in the heater matrix device.

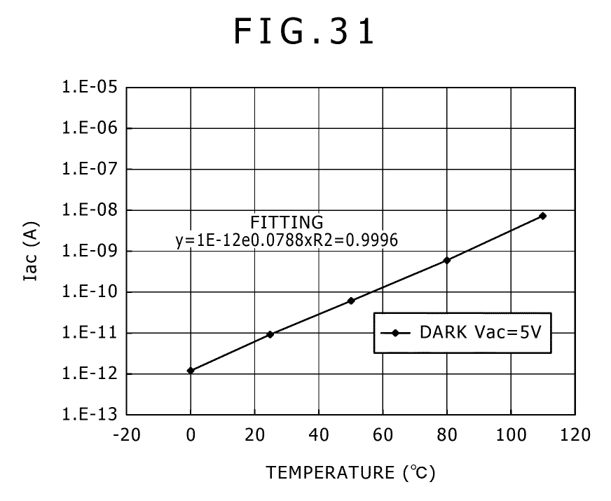

FIG. 31 is a graph showing the dependence of dark current on temperature.

This characteristic can be used to determine temperature from the detected current.

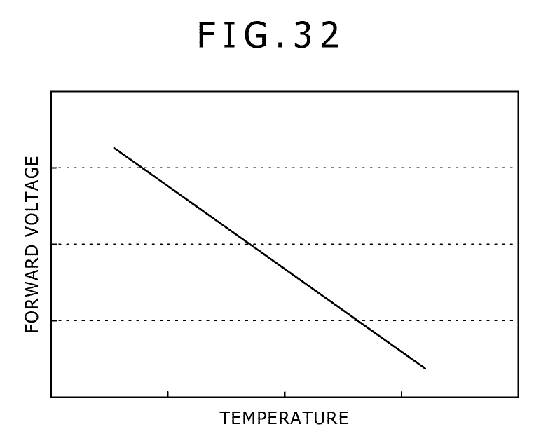

When the PIN diode D211 is given a certain forward current I_{det}, it produces a forward voltage which is related with temperature as shown in FIG. 32.

That is, the forward voltage changes linearly with temperature and hence the forward voltage of the temperature sense line TSL connected to the PIN diode D211 gives the information about temperature.

The following is a description of the fluorescence detecting matrix device.

### <Fluorescence Detecting Matrix Device>

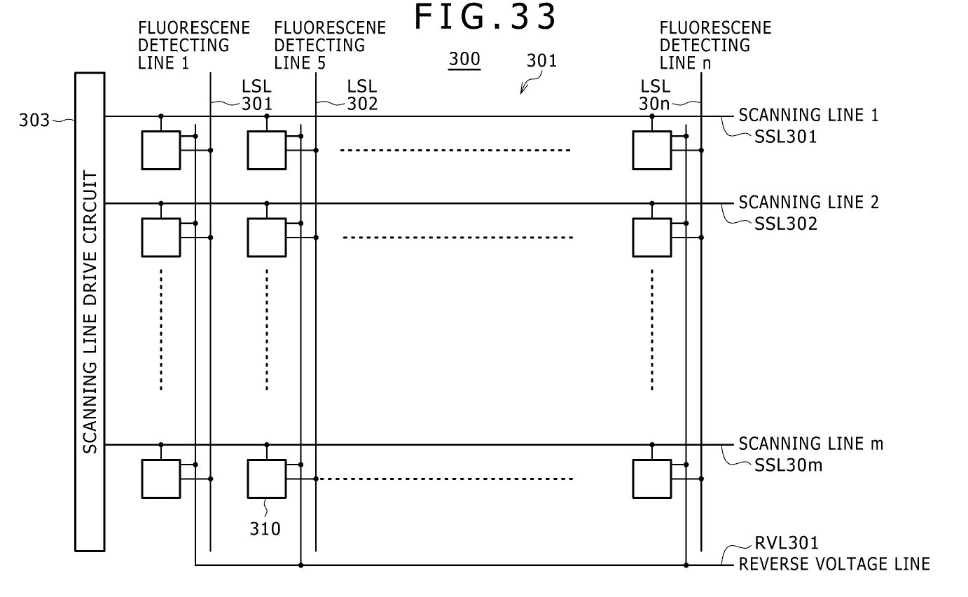

FIG. 33 is a schematic diagram showing the structure of the fluorescence detecting matrix device according to the embodiment of the present invention.

The fluorescence detecting matrix device 300 shown in FIG. 33 consists of the cell array 301 of fluorescence detecting units 310 arranged in an m X n matrix pattern, the scanning line driving circuit (WSDRV) 303, the reverse voltage line RVL301, the fluorescence detecting lines LSL301 . . . LSL30n, and the scanning lines SSL301 . . . SSL30m which select the detecting unit 310 and transfer the detecting signal from the fluorescence detecting unit 310 to the fluorescence detecting lines LSL301 . . . LSL30n.

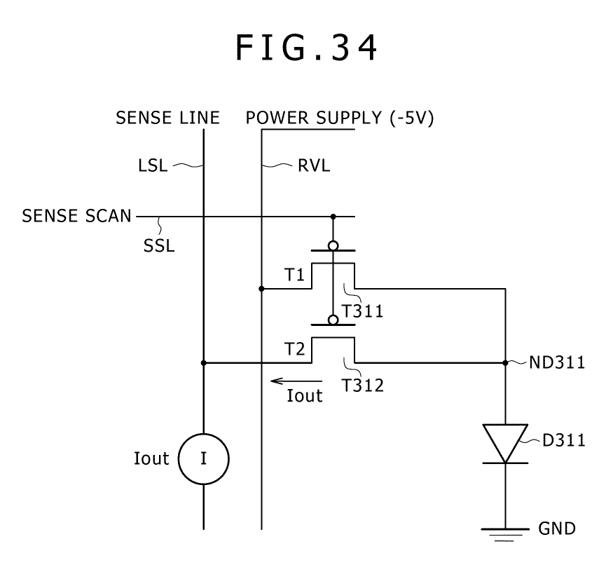

FIG. 34 is a circuit diagram showing the structure of the fluorescence detecting unit according to the embodiment of the present invention.

The fluorescence detecting unit 310 shown in FIG. 34 consists of the PIN diode D311, the p-channel transistors T311 and T312 which function as switches, and the node ND311.

The PIN diode D311 has its anode connected to the node D311 and its cathode connected to the ground potential GND.

The transistor T311 has its source and drain connected to the node ND311 and the reverse voltage line RVL, respectively. The transistor T312 has its source and drain connected to the node DN311 and the fluorescence sense line LSL.

The transistors T311 and T312 have their gates connected in common to the scanning line SSL.

The transistors T311 and T312 turn on or off when the scanning line SSL is at a low level or at a high level, respectively.

The fluorescence detecting unit 310 work in the following way.

When the reverse voltage line RVL is connected to the negative voltage source and the scanning line SSL is at a low level, the PIN diode D311 is reverse-biased by the negative voltage applied to the reverse voltage line RVL, and the reverse current IR flows.

This reverse current Iₒᵤₜ is detected by the fluorescence detecting line LSL. In this way fluorescence is detected.

The following is a description of the heater temperature detecting matrix device.

### <Heater Temperature Detecting Matrix Device>

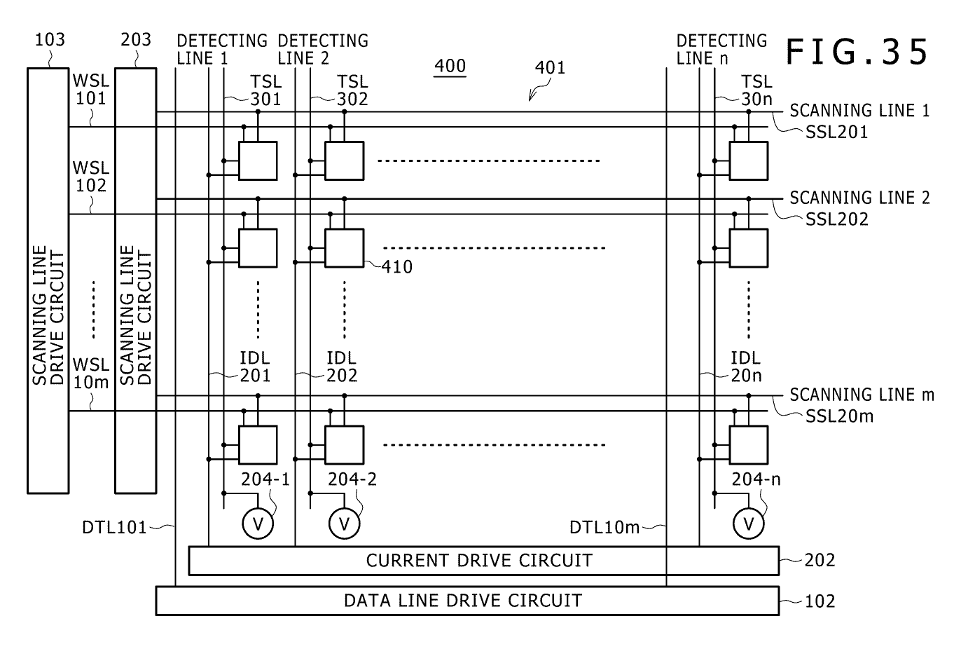

FIG. 35 is a schematic diagram showing the structure of the heater temperature detecting matrix device according to the embodiment of the present invention.

The heater temperature detecting matrix device 400 shown in FIG. 35 is a combination of the heater matrix device 100 shown in FIG. 15 and the temperature detecting matrix device 200 shown in FIG. 29. Therefore, the same symbols are applied to those components in FIG. 35 which are equivalent to those components in FIGS. 15 and 29, for easy understanding.

The heater temperature detecting matrix device 400 shown in FIG. 35 includes the cell array 401 of heater temperature detecting units 410 arranged in an m×n matrix pattern, the data line driving circuit (DTDRV) 102, the scanning line driving circuit (WSDRV) 103, the data lines DTL101 . . . DTL10m that supply information about the amount of heat generation to the heater unit 110, the scanning lines WSL101 . . . WSL10m which select the heater unit 110, write information about the amount of heat generation, and flow current in response to the written information about the amount of heat generation, the current driving circuit (IDRV) 202, the scanning line driving circuit (WSDRV) 203, the voltage detectors (V) 204-1 . . . 204-n, the current drive lines IDL201 . . . IDL20m, the temperature sense lines TSL201 . . . TSL20m, and the scanning lines SSL201 . . . SSL20m which select the temperature detecting unit 210 and transfer the detection signal of the temperature detecting unit 210 to the temperature detecting lines TSL201 . . . TSLL20m.

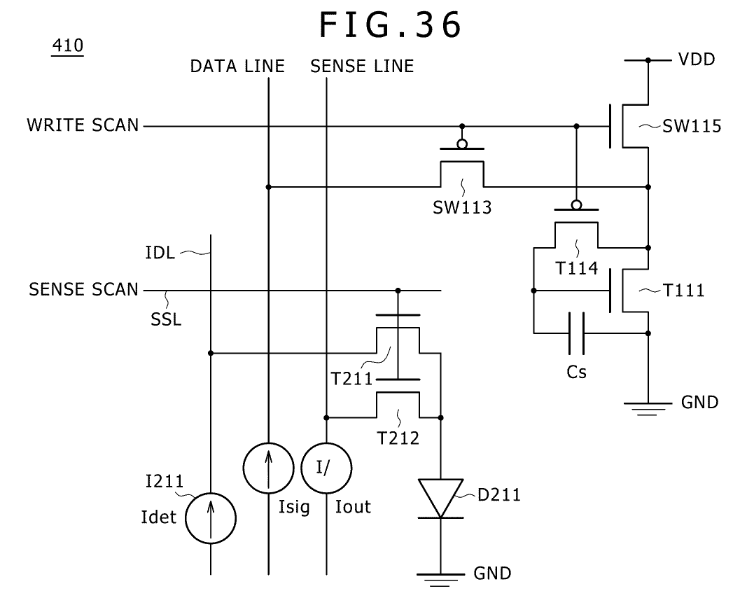

FIG. 36 is a circuit diagram showing the structure of the heater temperature detecting unit according to the embodiment of the present invention.

The heater temperature detecting unit 410 shown in FIG. 36 includes the heater unit shown in FIG. 23 and the temperature detecting unit 210 shown in FIG. 30.

Therefore, the same symbols are applied to those components in FIG. 36 which are equivalent to those components in FIGS. 15 and 30, for easy understanding.

The heater temperature detecting matrix device 400 shown in FIG. 35 senses the amount of actual heat generation after written as information about the amount of heat generation by current copier, so that it is capable of controlling and correcting the temperature by sensing dark current by means of the PIN diode for current copier and the written amount of information about the amount of heat generation.

In this case, the PIN diode D211 detects temperature by relation between the current of the heater unit 110 and the voltage in response to current flowing through the PIN diode of the temperature detecting unit 210.

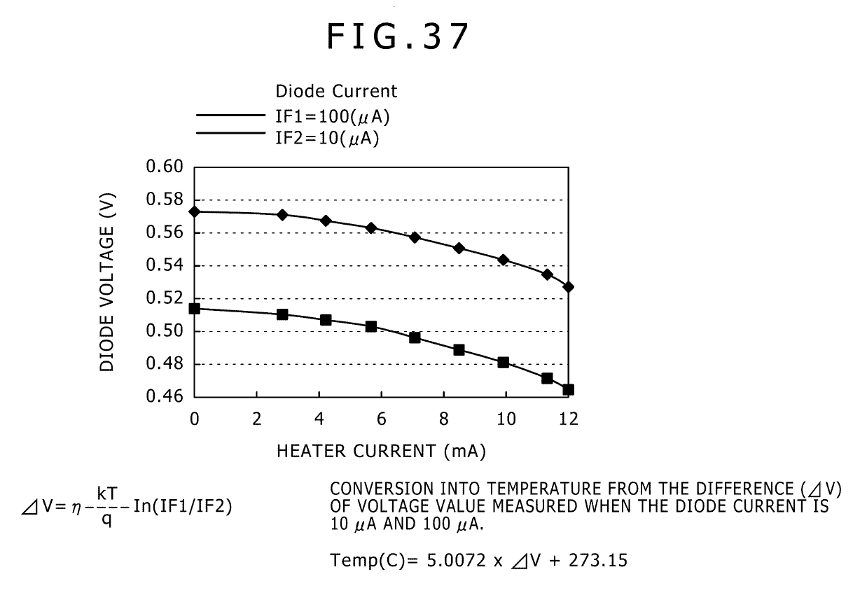

FIG. 37 is a graph showing the relation between the current of the heater unit and the voltage detected in response to current flowing through the PIN diode of the temperature detecting unit.

In FIG. 37, the abscissa represents the heater current and the ordinate represents the voltage of the diode.

In FIG. 37, IF1 denotes the voltage corresponding to the diode current of 10 μA, and IF2 denotes the voltage corresponding to the diode current of 100 μA.

Temperature can be obtained (by conversion) from the difference in voltage given by the following equation when the diode current is 10 μA and 100 μA.

### [Equation 6]

ΔV=η(kT/q)ln(IF1/IF2) (6)

Temp(C)=5.0072×ΔV+273.15

The following is a description of the temperature fluorescence detecting matrix device.

### <Temperature Fluorescence Detecting Matrix Device>

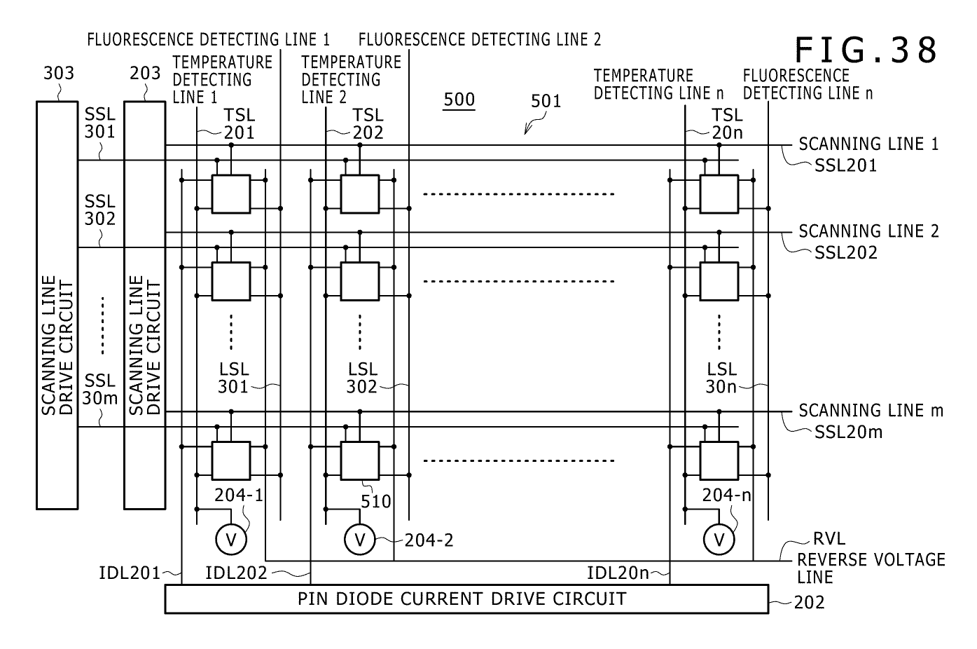

FIG. 38 is a schematic diagram showing the structure of the temperature fluorescence detecting matrix device according to the embodiment of the present invention.

The temperature fluorescence detecting matrix device 500 shown in FIG. 38 is a combination of the temperature detecting matrix device 200 shown in FIG. 29 and the fluorescence detecting matrix device 300 shown in FIG. 33. Therefore, the same symbols are applied to those components in FIG. 38 which are equivalent to those components in FIGS. 29 and 33, for easy understanding.

The temperature fluorescence detecting matrix device 500 shown in FIG. 38 consists of the cell array 501 of temperature fluorescence detecting units 510 arranged in an m X n matrix pattern, the current driving circuit (IDRV) 202, the scanning line driving circuit (WSDRV) 203, the voltage detectors (V) 204-1 . . . 204-n, the current driving lines IDL201 . . . IDL20m, the temperature sense lines TSL-201 . . . TSL20m, the scanning lines SSL201 . . . SSL20m which select the detecting unit 210 and transfer the detected signals of the temperature detecting unit 210 to the temperature sense lines TSL201 . . . TSL20m, the scanning lines SSI301 . . . SSL30n to select the fluorescence detecting unit 210, the scanning line driving circuit (WSDRV) 303, the reverse voltage line RVL301, and the fluorescence detecting lines LSL301 . . . LSL30n.

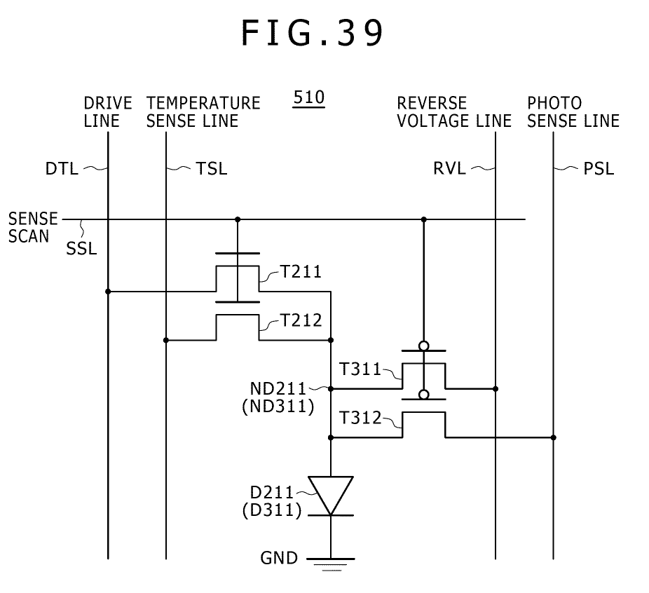

FIG. 39 is a circuit diagram showing the structure of the temperature fluorescence detecting unit according to the embodiment of the present invention.

The temperature fluorescence detecting unit 510 shown in FIG. 39 is a combination of the PIN diode D211 and the node ND211 of the temperature detecting unit 210 shown in FIG. 30 and the PIN diode D311 and the node ND311 of the fluorescence detecting unit 310 shown in FIG. 34. Therefore, the same symbols are applied to those components in FIG. 39 which are equivalent to those components in FIGS. 30 and 34, for easy understanding.

The temperature fluorescence detecting unit 510 includes one PIN diode D211 (D311), two n-channel transistors T211 and T212, and two p-channel transistors T311 and T312.

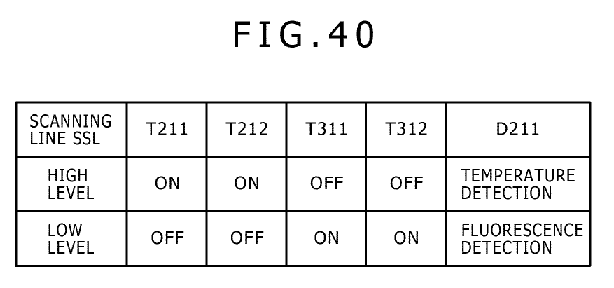

FIG. 40 shows how the temperature fluorescence detecting unit according to the embodiment of the present invention performs temperature detection and fluorescence detection depending on whether the transistors as switches turn on and off.

The scanning line SSL receives the switch signal which periodically changes from high level to low level and vice versa. The n-channel transistors T211 and T212 and the p-channel transistors T311 and T312 are connected in common to the scanning line SSL.

Thus, when the scanning line SSL is at a high level, the transistors T211 and T212 turn on and the transistors T311 and T312 turn off.

On the other hand, when the scanning line SSL is at a low level, the transistors T211 and T212 turn off and the transistors T311 and T312 turn on.

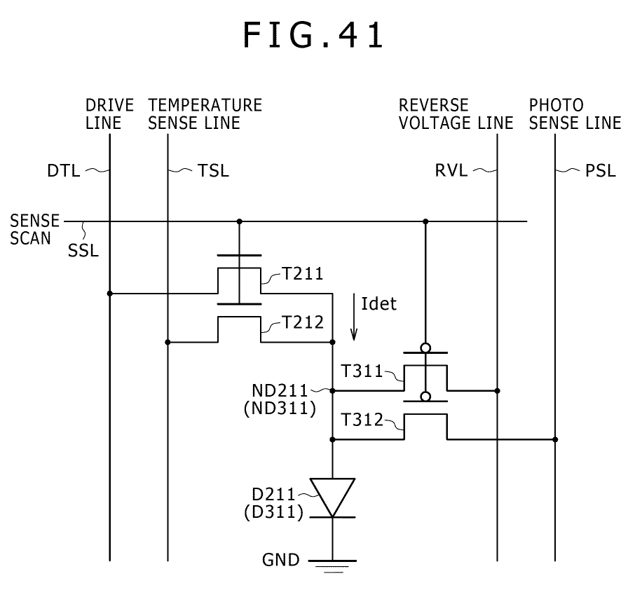

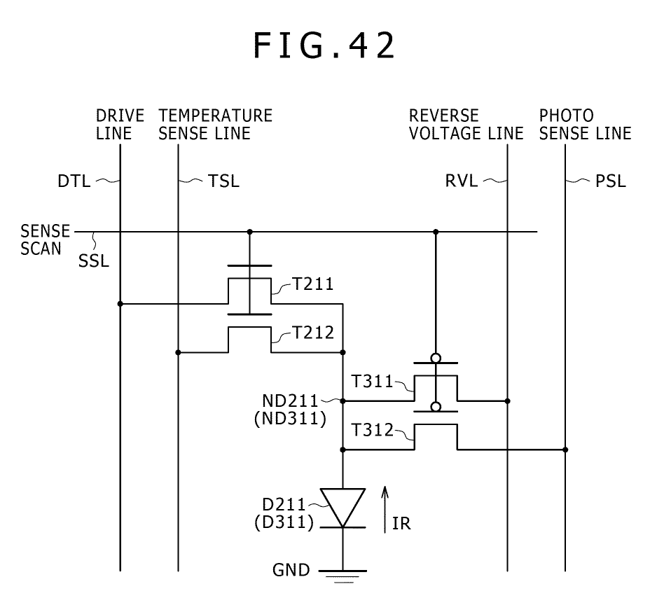

FIG. 41 is a diagram illustrating how temperature detection is performed by the temperature fluorescence detecting unit according to the embodiment of the present invention. FIG. 42 is a diagram illustrating how fluorescence detection is performed by the temperature fluorescence detecting unit according to the embodiment of the present invention.

At the time of temperature detection, connection is made with the current source I211 that supplies current I_{det} to the current drive line IDL. When the scanning line SSL is at a high level, a forward current I_{det} flows from the current source I211 connected to the current drive line IDL to the PIN diode D211. At the same time, the voltage detector 204 is connected to the temperature sense line TSL so that the forward voltage that occurs in the PIN diode D211 is detected.

There is a relationship as shown in FIG. 33 between the temperature and the forward voltage that occurs across the PIN diode D211 when a certain forward current I_{det} flows through the PIN diode D211.

In other words, there is a linear relationship between the forward voltage and the temperature, and the temperature information can be obtained by detecting the forward voltage of the temperature sense line TSL connected to the PIN diode D211.

At the time of fluorescence detection, the negative voltage source is connected to the reverse voltage line RVL, so that, when the scanning line SSL is at a low level, the PIN diode D311 is reverse-biased by the negative voltage applied to the reverse voltage line RVL and the reverse current IR flows as shown in FIG. 42.

This reverse current Iₒᵤₜ is detected through the fluorescence detecting line LSL to detect fluorescence.

The temperature fluorescence matrix device 500 shown in FIG. 38 works in such a way that the scanning line driving circuit 203 put the scanning lines SSL201 . . . SSL20m sequentially at a high level and, in synchronism with it, the current driving line driving circuit 202 applies a constant current to the current driving lines IDL201 . . . IDL20n and the voltage of the temperature sense line TSL201 . . . TSL20n is monitored, so that the temperature information can be detected row by row for each PIN diode D211.

After temperature detection is completed, the scanning lines are sequentially put to a low level, so that each PIN diode D211 is given a reverse voltage and the fluorescence information can be detected row by row for each PIN diode D211.

In this way each unit detects temperature and fluorescence alternately.

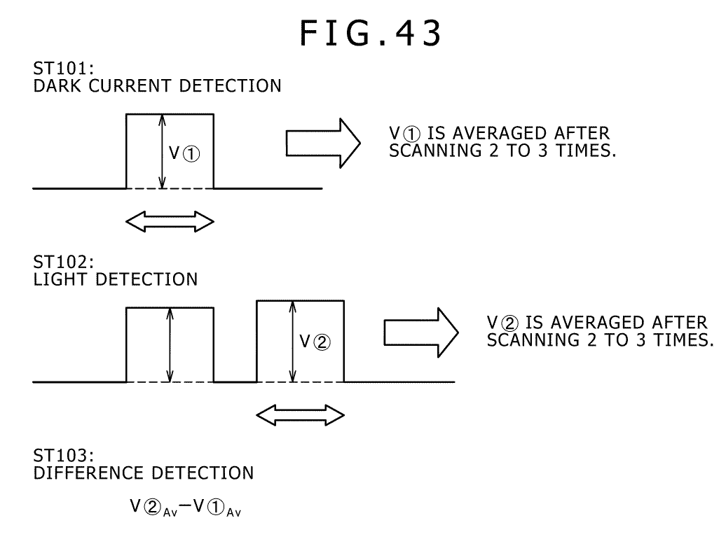

Incidentally, the fluorescence detection is accomplished in such a way that the dark current of the PIN diode D2111 (D311) is detected first, the detected value is binarized to give V1, and an average of V1 is obtained after scanning two or three times, as shown in FIG. 43. (ST101)

Then, the fluorescence detection mentioned above is accomplished, the detected value is binarized to give V2, and an average of V2 is obtained after scanning two or three times. (ST102)

The difference between V2 and V1 is obtained. (T103)

This procedure allows accurate fluorescence detection.

The following is a description of the heater temperature fluorescence detecting matrix device.

### <Heater Temperature Fluorescence Detecting Matrix Device>

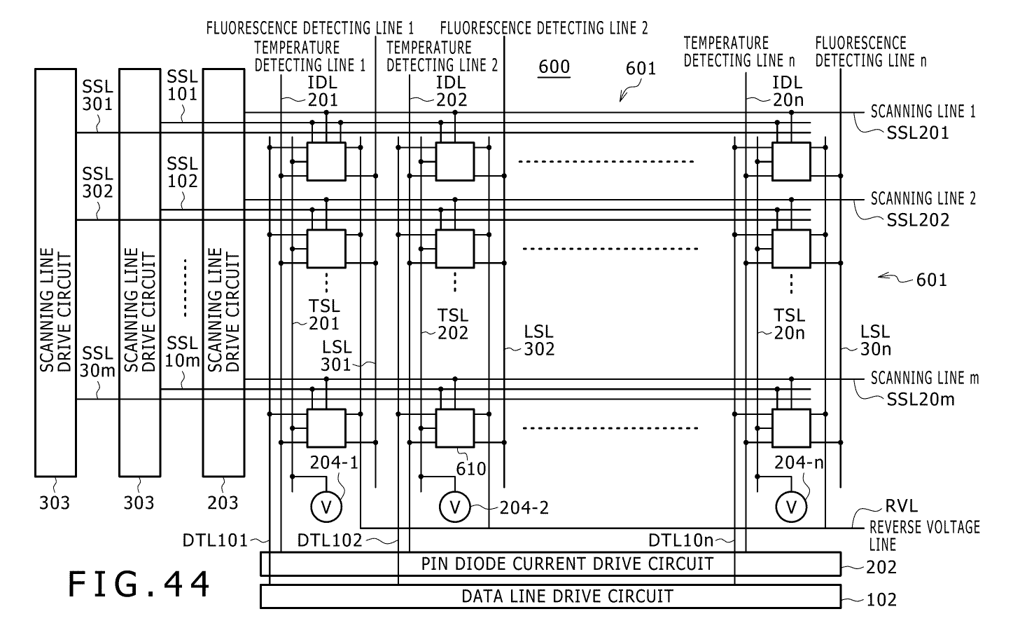

FIG. 44 is a schematic diagram showing the structure of the heater temperature fluorescence detecting matrix device according to the embodiment of the present invention.

The heater temperature fluorescence detecting matrix device 600 shown in FIG. 44 is a combination of the heater matrix device 100 shown in FIG. 15, the temperature detecting matrix device 200 shown in FIG. 30, and the fluorescence detecting matrix device 300 shown in FIG. 35. Therefore, the same symbols are applied to those components in FIG. 43 which are equivalent to those components in FIGS. 15, 30, and 33, for easy understanding.

The heater temperature fluorescence detecting matrix device shown 600 shown in FIG. 44 includes the cell array 601 of heater temperature fluorescence detecting units 610 arranged in an m×n matrix pattern, the data driving circuit (DTDRV) 102, the scanning line driving circuit (WSDRV) 103, the data lines DTL101 . . . DTL10m that give the information about the amount of heat generation to the heater unit 110, the scanning lines WSL101 . . . WSL10m which select the heater unit 210, write the information about the amount of heat generation, and flow current in response to the information about the amount of heat generation which has been written, the current driving circuit (IDRV) 202, the scanning line driving circuit (WSDRV) 203, the voltage detectors (V) 204-1 . . . 204-n, the current drive lines IDL201 . . . IDL20m, the temperature sense lines TSL201 . . . TSL20m, the scanning liens SSL201 . . . SSL20m which select the temperature detecting unit 210 and transfer the signals detected by the temperature detecting unit 210 to the temperature detecting lines TSL201 . . . TSL20n, the current driving circuit (IDTC) 302, and scanning line driving circuit (WSDRV) 303, the reverse voltage line RVL301, and the fluorescence detecting lines LSL301 . . . LSL30m.

The foregoing structure may be modified such that the data line driving circuit 102 and the current driving circuit 202 function in common.

In this case the data line DTL and the temperature sense line TSL function in common.

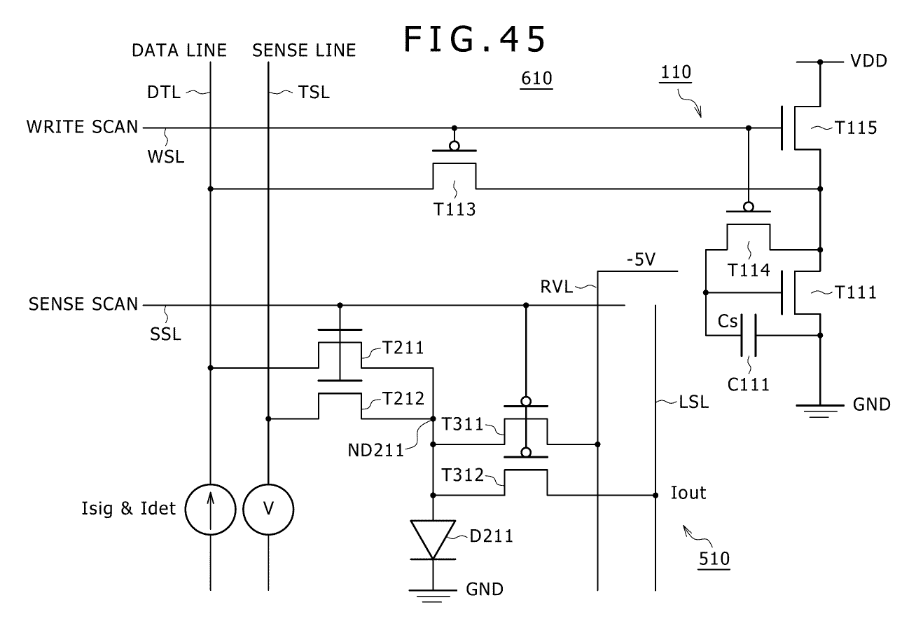

FIG. 45 is a circuit diagram showing the structure of the heater temperature fluorescence detecting unit according to the embodiment of the present invention.

The heater temperature fluorescence detecting unit 610 shown in FIG. 45 consists of the heater unit 110 shown in FIG. 23 and the temperature fluorescence detecting unit 510 shown in FIG. 39. Therefore, the same symbols are applied to those components in FIG. 45 which are equivalent to those components in FIGS. 23 and 29, for easy understanding.

In this embodiment, the data line DTL and the temperature detecting line TSL function in common.

The heater temperature fluorescence detecting matrix devic3 600 shown in FIG. 44 senses the amount of actual heat generation by using current copier after writing as the information about the amount of heat generation, so that it senses the dark current by the PIN diode for the written information about the amount of heat generation for current copier. In this way it is possible to correct the temperature control.

And, by sensing the current that occurs when fluorescence is received, it is possible to detect the reaction of amplification.

To be specific, it is possible to detect in real time the reaction of amplification in terms of the amount of fluorescence by the PIN diode D211 which is the temperature detecting device by using the detection of fluorescence as the signal of detection of amplification reaction in the stage of feeding back in real time the control of heat generation by the circuit composed of the current copier (heater unit) and the temperature detecting unit.

As mentioned above, the heat control matrix device applicable to the reactor for DNA amplification produces the following effects.

It is possible to control the temperature of individual wells by active matrix control and hence it is possible to perform comprehensive gene analysis in a short time.

It is possible to obtain the accurate amount of heat generation by feedback mechanism owing to the temperature detecting circuit even though the semiconductor elements vary in characteristics or have temperature characteristics, and this leads to efficient PCR control.

It is possible to obtain the accurate amount of heat generation by feedback mechanism owing to the temperature detecting circuit even though the semiconductor elements change with time in characteristics, and this leads to the highly reliable PCR control device.

Having the function to suspend the action of heat generation by each scanning line, it is possible to lower the temperature easily and rapidly, and being able to control the duration of heating, it is easy to control minute heat generation.

When the information about heat generation is written, it accurately senses the actual amount of heat generation and corrects the written amount of heat generation, so that it offers the accurate amount of heat generation.

It is possible to detect fluorescence as the signal of amplification reaction by using the circuit identical with the temperature detecting circuit for temperature sensing.

Thus, the reactor according to this embodiment permits temperature control to be performed on wells accurately and individually. This reactor will be used in any application area where reactions with accurate temperature control are required. It is suitable particularly for the PCR device for gene amplification reaction.

The present application contains subject matter related to that disclosed in Japanese Priority Patent Application JP 2008-105841 filed in the Japan Patent Office on Apr. 15, 2008, the entire content of which is hereby incorporated by reference.

It should be understood by those skilled in the art that various modifications, combinations, sub-combinations and alterations may occur depending on design requirements and other factors insofar as they are within the scope of the appended claims or the equivalents thereof.

1. A reactor, comprising:
a plurality of reaction regions, wherein a first subset of the plurality is arranged in a heating portion and a second subset is arranged in a cooling portion;
a plurality of heating elements, each arranged in one of said reaction regions in said heating portion; and
cooling elements to cool said reaction regions in said cooling portion, wherein
each said heating element comprises
a heater,
a temperature detecting element,
detection means for detecting temperature information from said temperature detecting element, and
temperature control means for controlling a temperature of said heater according to the detected temperature information,
said temperature control means performing
processing for a temperature cycle that includes a first temperature holding control for a denature treatment,
processing for a second temperature holding control to cool from the denature treatment to an annealing treatment and also for the annealing treatment,
processing for a first temperature rise control for a first heating from the annealing treatment to an extension treatment,
processing for a third temperature holding control for the extension treatment, and
processing for a second temperature rise control for a second heating from the extension treatment to the denature treatment.

2. A reactor, comprising:
a plurality of reaction regions, wherein a first subset of the plurality is arranged in a heating portion and a second subset is arranged in a cooling portion;
a plurality of heating elements, each arranged in one of said reaction regions in said heating portion; and
cooling elements to cool said reaction regions in said cooling portion, wherein
each said heating element comprises
a heater,
a temperature detecting element,
detection means for detecting temperature information from said temperature detecting element, and
temperature control means for controlling a temperature of said heater according to the detected temperature information,
said temperature control means performs processing including
a first temperature holding control for a denature treatment,
a temperature down control to cool from the denature treatment to an annealing treatment,
a second temperature holding control for the annealing treatment and an extension treatment, and
a temperature rise control to heat from the extension treatment to the denature treatment.

3. The reactor as defined in claim 1, wherein said processing performed by the temperature control means further includes:
detecting temperature from said temperature detecting element;
calculating an amount of heater control;
controlling said heater; and
controlling said cooling element.

4. The reactor as defined in claim 2, wherein said processing performed by the temperature control means further includes:
detecting temperature from said temperature detecting element;
calculating an amount of heater control;
controlling said heater; and controlling the cooling element.

5. The reactor as defined in claim 1, wherein said processing performed by the temperature control means further includes:
detecting temperature from said temperature detecting element;
controlling current to be applied to said temperature detecting element; and
converting a voltage of said temperature detecting element by means of an analog-digital converter.

6. The reactor as defined in claim 2, wherein said processing performed by the temperature control means further includes:
detecting temperature from said temperature detecting element;
controlling current to be applied to said temperature detecting element; and
converting a voltage of said temperature detecting element by means of an analog-digital converter.
